(19) United States
(12) Patent Application Publication
(10) Pub. No.: US 2023/0123431 A1
(43) Pub. Date: Apr. 20, 2023

(54) METABOLIC ENGINEERING FOR PRODUCTION OF LIPOIC ACID

(71) Applicant: National University of Singapore, Singapore (SG)

(72) Inventors: Matthew Wook Chang, Singapore (SG); Binbin Chen, Singapore (SG); Jee Loon Foo, Singapore (SG); Hua Ling, Singapore (SG)

(73) Assignee: National University of Singapore, Singapore (SG)

(21) Appl. No.: 17/905,592

(22) PCT Filed: Mar. 2, 2021
(86) PCT No.: PCT/SG2021/050103

§ 371 Date: Sep. 2, 2022

(30) Foreign Application Priority Data
Mar. 2, 2020 (SG) 10202001884X

(51) Int. Cl. C12N 15/52; C12N 15/81; C12P 7/40; C12P 11/00; C12N 1/16

(52) U.S. Cl. CPC C12N 15/52; C12N 15/81; C12P 7/40; C12P 11/00; C12N 1/16; C12Y 208/01008

(57) ABSTRACT

The present invention provides for a method to increase the free lipoic acid production in an isolated genetically engineered bacteria or yeast cell. The method involves culturing in a cysteine supplemented culture medium the engineered bacteria or yeast that is transformed with a recombinant expression vector encoding polynucleotide molecules that results in the overexpression of the following genes that are linked to at least one promoter: (1) substrate protein (e.g. Gcv3p); (2) octanoyltransferase or lipoyl synthase; (3) cofactor S-adenosyl methionine synthase; and (4) lipoamidase. The invention also relates to the engineered bacteria or yeast cell thereof.

### FIELD OF THE INVENTION

The present invention provides a genetically engineered bacteria or yeast cell capable of enhanced production of free lipoic acid, recombinant vectors, and methods for the production of free lipoic acid. More particularly, the free lipoic acid is R-lipoic acid.

### BACKGROUND OF THE INVENTION

Lipoic acid is an essential cofactor required for several key enzymes involved in aerobic metabolism and the glycine cleavage system in most organisms (Cronan et al., Advances in Microbial Physiology, RK. Poole, Editor, Academic Press. 103-146 (2005); Cronan, Microbiology and Molecular Biology Reviews 80: 429-450 (2016)). It can be used as an antioxidant for dietary supplementation due to its ability to bind directly or indirectly with free radicals (Croce et al., Toxicology in Vitro 17: 753-759 (2003)). Furthermore, findings from clinical trials have shown that lipoic acid can increase insulin sensitivity, which supports its application as an anti-diabetic drug (Lee et al., Biochemical and Biophysical Research Communications 443: 885-891 (2005)). Lipoic acid was also shown to inhibit the proliferation of breast tumor cells, indicating its potential application as an anti-cancer drug (Li et al., Genetics and Molecular Research 14: 17934-17940 (2015)). Currently, lipoic acid is obtained mainly through chemical synthesis processes, which conventionally generates equal amounts of the two enantiomeric R and S forms of lipoic acid (Balkenhohl and Paust, Zeitschrift for Naturforschung Section B-a Journal of Chemical Sciences 54: 649-654 (1999); Ide et al., Journal of Functional Foods 5: 71-79 (2013)). However, in biological systems, lipoic acid exists solely in the R form; S-lipoic acid is a by-product during chemical synthesis. Therefore, R-lipoic acid in general shows bioactivity superior to S-lipoic acid, and in some cases, S-lipoic acid is detrimental to health. For example, R-lipoic acid was shown to protect the lens in eyes from forming cataract, while S-lipoic acid showed the reverse effect by potentiating deterioration of the lens (Kilic et al., Biochem Mol Biol Int 37: 361-370 (1995)). Thus, it is beneficial to obtain R-lipoic acid in the enantiomerically pure form to maximize the health effects of lipoic acid and prevent potential side effects caused by S-lipoic acid. Yet, chiral separation and asymmetric synthesis methods used to attain pure R-lipoic acid lead to wastage of the S form of lipoic acid or precursors of undesired chirality (U.S. Pat. Nos. 5,281,722A; 6,670,484 B2; 6,864,374 B2; Purude et al., Tetra-hedron-Asymmetry 26: 281-287 (2015)), hence reducing the efficiency of resource utilization in synthesizing the compound. Moreover, compared to racemic lipoic acid synthesis, these procedures for preparing pure R-lipoic acid lengthen the production process, and require additional reagents and solvents, which incur higher manufacturing costs and greater impact on the environment. In view that chemical synthesis of R-lipoic acid also involves toxic reagents and catalysts, and entails many steps, biological engineering of microbial cell factories for production of free R-lipoic acid presents an attractive avenue for obtaining enantiomerically pure R-lipoic acid in a sustainable and environmentally-friendly manner. Bacterial production of lipoic acid through metabolic engineering has been shown in bacteria, including Escherichia coli, Pseudomonas reptilivora, Listeria monocytogenes and Bacillus subtilis, and so on (Ji et al., Biotechnology Letters 30: 1825-1828 (2008); Moon et al., Applied Microbiology and Biotechnology 83: 329-337 (2009); Christensen et al., Mol Microbiol 80: 350-363 (2011); Storm, CurrPharm Des 18: 3480-3489 (2012); Sun et al., PLoS one 12: e0169369-e0169369 (2017)). The lipoic acid biosynthesis and protein lipoylation pathways are most well-studied in E. coli over the past two decades. There are two complementary pathways for lipoic acid biosynthesis and protein lipoylation in E. coli: (i) de novo biosynthesis pathway where endogenous octanoic acid is attached to apo-proteins by LipB, followed by sulfur insertion by LipA, and (ii) scavenging pathway where exogenous lipoic acid or octanoic acid is transferred to unlipoylated apo-form of proteins by Lp1A (Sun et al., PLoS one 12: e0169369-e0169369 (2017)).

Compared to bacteria, Saccharomyces cerevisiae, a model yeast strain, offers a number of advantages for biochemical production due to its inherent abilities to withstand lower temperature, pH changes and phage attack (Chen et al., Metabolic Engineering 31: 53-61 (2015); Jin et al., Biotechnol Bioeng 113: 842-851 (2016); Foo et al., Biotechnology and Bioengineering 114: 232-237 (2017)). Importantly, unlike E. coli, yeast lacks a lipoic acid scavenging pathway that binds free lipoic acid to proteins via an ATP- and energy-expending process (Booker, Chemistry & Biology 11: 10-12 (2004)). Hence, S. cerevisiae inherently does not consume free lipoic acid, which is a beneficial characteristic that allows accumulation of our target compound, i.e. free R-lipoic acid.

In yeast, there are three well-known lipoate-dependent enzyme systems: glycine cleavage system (GCV), α-ketoglutarate dehydrogenase (KGDC) and pyruvate dehydrogenase (PDH) (Schonauer et al., Journal of Biological Chemistry 284: 23234-23242 (2009)). GCV is involved in the cleavage of glycine to ammonia and C1 units, which is essential for utilization of glycine as a sole source of nitrogen (Sinclair and Dawes, Genetics 140: 1213-1222 (1995); Piper et al., FEMS Yeast Research 2: 59-71 (2002)). KGDC catalyzes the oxidative decarboxylation of 2-oxoglutarate to succinyl-CoA, a precursor of several amino acids and the source of succinate, the entry point to the respiratory chain (Repetto and Tzagoloff, Molecular and Cellular Biology 11: 3931-3939 (1991)). PDH catalyzes the oxidative decarboxylation of pyruvate, thereby linking cytosolic glycolysis and mitochondrial respiration (Boubekeur et al., Journal of Biological Chemistry, 274(30): 21044-21048 (1999)). Gcv3p, Kgd2p and Lat1p are the lipoate-bound subunits of GCV, KGDC and PDH respectively (Nagarajan and Storms, Journal of Biological Chemistry 272: 4444-4450 (1997)). Different from E. coli, lipoic acid synthesis and attachment to target proteins are less well-understood in yeast. To form lipoylated Gcv3p, Kgd2p and Lat1p, a two-step conversion has been hypothesized for lipoic acid synthesis and protein attachment in yeast mitochondria (Hermes and Cronan, Yeast 30: 415-427 (2013)). Lip2p and Lip3p were demonstrated to encode octanoyltransferases that utilize octanoyl-ACP or octanoyl-CoA to attach an octanoyl group to the apo-form of lipoate-dependent proteins (Stuart et al., FEBS Letters 408: 217-220 (1997); Marvin et al., FEMS Microbiology Letters 199: 131-136 (2001); Hermes and Cronan, Yeast 30: 415-427 (2013)). A lipoyl synthase Lip5p catalyzes the insertion of two sulfurs into the octanoate carbon chain (Sulo and Martin, Journal of Biological Chemistry 268: 17634-17639 (1993)). Ultimately, lipoic acid is bound to Gcv3p, Kgd2p and Lat1p via an amide linkage between its carboxyl group and the epsilon amino group of a lysine residue of the proteins (Sulo and Martin, Journal of Biological Chemistry 268: 17634-17639 (1993)). Interestingly, it has been discovered that Lip2p and Lip5p are required for lipoylation of all three proteins while Lip3p is required for lipoylation of Kgd2p and Lat1p but not Gcv3p (Hermes and Cronan, Yeast 30: 415-427 (2013)). To release free lipoic acid from lipoate-bound proteins, lipoamidase from Enterococcus faecalis (EfLPA), a member of the Ser-Ser-Lys family of amidohydrolases was isolated and characterized (Jiang and Cronan, Journal of Biological Chemistry 280: 2244-2256 (2005)). This enzyme has been demonstrated to liberate free lipoic acid from lipoic acid-bound H protein of GCV, and E2 subunit of KGDC and PDH from E. coli (Spalding and Prigge, PLoS one 4: e7392 (2009)). While functional heterologous expression of EfLPA has been demonstrated in bacterial hosts, the activity of EfLPA in yeast is, to the inventor's knowledge, unknown.

There is a need to improve methods for the production of free R-lipoic acid. Therefore, S. cerevisiae as a potential production host for free R-lipoic acid biosynthesis was investigated. Hereafter, lipoic acid specifically refers to R-lipoic acid.

### SUMMARY OF THE INVENTION

EfLPA has been demonstrated to liberate free lipoic acid from lipoic acid-bound H protein of GCV, and E2 subunit of KGDC and PDH from E. coli (Spalding and Prigge, PLoS one 4:e7392 (2009)). The inventors employed metabolic engineering strategies to improve lipoic acid production. First, the availability of lipoate-bound proteins in yeast was confirmed and they were then characterized through liquid chromatography-tandem mass spectrometry (LC-MS/MS). The in vitro activity of EfLPA was determined in order to validate its functional expression and to select a suitable lipoylated protein as the target substrate for EfLPA. To develop a free lipoic acid-producing strain, EfLPA was modified for translocation to the mitochondria, where lipoylated proteins reside. Finally, to enhance the lipoic acid production, the selected substrate protein (i.e. Gcv3p), catalytic enzymes (i.e. Lip2p and Lip5p), and cofactor regenerating enzymes (i.e. Sam1p and Sam2p) were overexpressed (FIG. 1). The proteomic analysis, enzyme characterization and metabolic engineering approaches collectively enabled unprecedented free lipoic acid production in S. cerevisiae to be accomplished In a first aspect, the present invention provides an isolated genetically engineered bacteria or yeast cell, wherein the bacteria or yeast cell has been transformed by at least one polynucleotide molecule; the at least one polynucleotide molecule comprising lipoic acid pathway genes which encode a octanoyltransferase, a lipoyl synthase, a protein substrate that is lipoylated, a lipoamidase and/or S-adenosylmethionine synthase, operably linked to at least one promoter, wherein at least one lipoic acid pathway gene is heterologous and said genetically engineered bacteria or yeast cell is capable of increased production of free lipoic acid compared to a non-transformed cell.

The protein substrate that is lipoylated may be any suitable substrate known in the art and may be selected from a group comprising Gcv3p, Lat1p and Kgd2p.

It would be understood that the S-adenosylmethionine synthase may be any suitable enzyme known in the art, preferably from a cell selected from a group comprising Kluyveromyces, Candida, Pichia, Yarrowia, Debaryomyces, Saccharomyces spp., and Schizosaccharomyces pombe. Preferably, the S-adenosylmethionine synthase is S-adenosylmethionine synthase 1 (Sam1) and/or S-adenosylmethionine synthase 2 (Sam2), more preferably Sam1 and Sam2 are from Saccharomyces cerevisiae.

In some embodiments, the lipoic acid pathway genes comprise LIP2 (octanoyltransferase), LIP5 (lipoyl synthase), GCV3 (H protein of the glycine cleavage system), LPA (lipoamidase), SAM1 and/or SAM2.

In some embodiments, the lipoic acid pathway genes are expressed in mitochondria.

In some embodiments, the lipoic acid pathway genes are expressed in the mitochondria by virtue of a mitochondrial targeting peptide (MTP). It was found that proteins such as Gcv3p, Lat1p and Kgd2p could be targeted to the mitochondria through their native MTP, whereas LPA, Sam1p and Sam2p could be targeted to the mitochondria using a non-native MTP, such as the MTP from yeast cytochrome c oxidase subunit IV.

In some embodiments, the mitochondrial targeting peptide (MTP) is from yeast cytochrome c oxidase subunit IV (COX4). In some embodiments the amino acid sequence of the COX4 MTP is 5′-MLSLRQSIRFFKPATRTLCSSRYLLQQKP-3′ (SEQ ID NO: 45).

In some embodiments, the yeast is selected from a group comprising Kluyveromyces, Candida, Pichia, Yarrowia, Debaryomyces, Saccharomyces spp., and Schizosaccharomyces pombe. Preferably, the yeast is Saccharomyces cerevisiae.

In some embodiments, said at least one promoter is a constitutive promoter.

In some embodiments the lipoamidase (LPA) is from Enterococcus faecalis, termed EfLPA. Preferably, the EfLPA gene is codon-optimized for expression in S. cerevisiae. If the EfLPA gene is used, it is preferred that the protein substrate that is targeted for lipoylation is Gcv3p.

In some embodiments, the lipoic acid pathway genes are expressed from one or more plasmids. Alternatively, expression cassettes encoding one or more of the heterologous lipoic acid pathway genes may be integrated into the genome using an integrative vector such as pIS385 described in Example 1. It would be understood that integration into the host DNA may provide permanent expression, whereas plasmid expression tends to be transient.

In some embodiments, at least one of said lipoic acid pathway genes is integrated into the bacteria or yeast genome.

In some embodiments, the LIP2, LIP5, GCV3, LPA, SAM1 and/or SAM2 genes respectively encode an amino acid sequence comprising the sequence set forth in SEQ ID NO: 1, SEQ ID NO: 3, SEQ ID NO: 5, SEQ ID NO: 7, SEQ ID NO: 9 and/or SEQ ID NO: 11. It would be understood that due to redundancy in the genetic code, a nucleic acid sequence may have less than 100% identity to a reference sequence and still encode the same amino acid sequence.

In some embodiments, the LIP2 gene comprises a polynucleotide sequence having at least 70% sequence identity, at least 80% sequence identity, at least 85% sequence identity, at least 90% sequence identity, at least 95% sequence identity, or 100% sequence identity to the sequence set forth in SEQ ID NO: 2; the LIP5 comprises a polynucleotide sequence having at least 70% sequence identity, at least 80% sequence identity, at least 85% sequence identity, at least 90% sequence identity, at least 95% sequence identity, or 100% sequence identity to the sequence set forth in SEQ ID NO: 4; the GCV3 gene comprises a polynucleotide sequence having at least 70% sequence identity, at least 80% sequence identity, at least 85% sequence identity, at least 90% sequence identity, at least 95% sequence identity, or 100% sequence identity to the sequence set forth in SEQ ID NO: 6; the LPA gene comprises a polynucleotide sequence having at least 70% sequence identity, at least 80% sequence identity, at least 85% sequence identity, at least 90% sequence identity, at least 95% sequence identity, or 100% sequence identity to the sequence set forth in SEQ ID NO: 8; the SAM1 gene comprises a polynucleotide sequence having at least 70% sequence identity, at least 80% sequence identity, at least 85% sequence identity, at least 90% sequence identity, at least 95% sequence identity, or 100% sequence identity to the sequence set forth in SEQ ID NO: 10 and/or the SAM2 gene comprises a polynucleotide sequence having at least 70% sequence identity, at least 80% sequence identity, at least 85% sequence identity, at least 90% sequence identity, at least 95% sequence identity, or 100% sequence identity to the sequence set forth in SEQ ID NO: 12.

In a second aspect, the invention provides a recombinant expression vector comprising one or more heterologous lipoic acid pathway genes operably linked to a promoter according to any aspect of the invention, wherein an expressed protein is located to the mitochondria.

In some embodiments, the promoter is a constitutive promoter.

In a third aspect, the invention provides a method of producing free lipoic acid in a genetically engineered cell, comprising the steps:
a) culturing a plurality of genetically engineered cells according to any aspect of the invention in a medium under conditions for lipoic acid biosynthesis, and
b) supplementing the medium with cysteine, wherein said genetically engineered cell is capable of increased production of free lipoic acid compared to a non-transformed cell.

In some embodiments, the medium is supplemented with cysteine at a concentration of at least 0.05 mg/ml, at least 0.1 mg/ml, at least 0.2 mg/ml, at least 0.5 mg/ml or in the range from 0.05 mg/ml to 0.7 mg/ml, preferably in the range 0.1 mg/ml to 0.4 mg/ml.

In some embodiments, the method further comprises isolating said free lipoic acid.

In a preferred embodiment, the cell is a bacterium or a yeast cell.

More preferably, the cell is Saccharomyces cerevisiae.

### BRIEF DESCRIPTION OF THE FIGURES

FIG. 1 shows a schematic view of the metabolic pathway for the production of lipoic acid in engineered S. cerevisiae. Apo-Gcv3p is a substrate protein while octanoyl and lipoyl-Gcv3p are the two intermediates in the lipoic acid producing pathway. Lipoyl-Gcv3p is the lipoic acid bound H subunit of glycine cleavage system (GCV). Lip2p and Lip5p work as catalyst enzymes. EfLPA is the cleaving enzyme for releasing lipoic acid. Sam2p is a cofactor regeneration enzyme required to regenerate the S-adenosyl methionine cofactor, as shown in the dotted box. Lip2p: octanoyltransferase; Lip5p: lipoyl synthase; EfLPA: lipoamidase from E. faecalis; Sam2p: S-adenosylmethionine synthase 2. All reactions are in mitochondria.

FIG. 2A-E shows the detection of product ions (b and y) in lipoyl/octanoyl-modified peptides. (A)-(C) show the calculated m/z of ions in MS/MS spectra of peptides with lipoic acid modification. (D)-(E) show the calculated m/z of ions in MS/MS spectra of peptides with octanoic acid modification. The sequences obtained are shown at the top of the table. Detected b ions are shown with *, while detected y ions are shown with ∧. “#” indicates the position of the amino acid in the sequence. 188.03 and 126.10 represent the masses of lipoyl and octanoyl, respectively.

FIG. 3A-E shows the detection of lipoyl/octanoyl-modified peptides. (A)-(C) show the MS/MS spectra of peptides with lipoic acid modification. Singly charged peptide (m/z=895.3918) of Gcv3p (A) and peptide (m/z=1021.4584) of Kgd2p (B) as well as doubly charged peptide (m/z=636.7529) of Lat1p (C) were detected with lipoic acid modification at position K102, K114 and K75 respectively. (D)-(E) show MS/MS spectra of peptides with octanoic acid modification. Singly charged peptide (m/z=833.4583) with octanoic acid modification at position S100 (D) and peptide (m/z=833.4628) with octanoic acid modification at position S103 (E) were detected. S: serine; V: valine; K: lysine; A: alanine; E: glutamic acid; T: threonine; D: aspartic acid; I: isoleucine; Q: glutamine; M: methionine; F: phenylalanine; Lipoyl: lipoyl modification; Octanoyl: octanoyl modification.

FIG. 4B-D shows the proposed mechanism for sulfur insertion of Gcv3p (A), and 3D protein structures of (B) Gcv3p, (C) lipoyl domain of KGD2 and (D) lipoyl domain of LAT1. Helix was shown in light blue, sheet in red and loop in purple. Surface of protein was show in grey. K stands for lysine residue while S stands for serine residue.

FIG. 5A-F shows GCV3, KGD2, LAT1 and EfLPA protein expression and lipoamidase activity of EfLPA towards GCV3 in vitro. (A) Expression of GCV3, KGD2, LAT1 and EfLPA. The expression of GCV3, KGD2, LAT1 and EfLPA were confirmed by western blot analysis. (B) LC-MS/MS chromatogram of extracted product from EfLPA and Gcv3p mixture. Peak of lipoic acid is indicated by an arrow at retention time 4.362 min. (C) LC-MS/MS spectrum of the single-charged ion of lipoic acid. Lipoic acid detected in (B) was further fragmented by MS/MS. (D) LC-MS/MS spectrum of the single-charged ion of lipoic acid standard reference. The precursor ions, 205.0360 for (C) and 205.0365 for (D), both marked with a diamond. m/z value of product ions were labelled. (E) GCMS chromatogram of extract from EfLPA and Gcv3p mixture. Trimethylsilylated lipoic acid (lipoyl-TMS) was detected at a retention time of 23.675 min. (F) GCMS spectrum of the lipoyl-TMS peak in (E) is shown in the top spectrum. It is identical to the bottom GCMS spectrum obtained using a trimethylsilylated lipoic acid authentic reference standard.

FIG. 6A-C shows subcellular localization of EfLPA and lipoic acid production in vivo. (A) Characterization of the mitochondria targeting peptide. Cells carrying EGFP fused with and without mitochondria signal peptide (mEGFP and EGFP) were harvested. Fluorescence figures were shown. (B) Subcellular localization of EfLPA. Proteins in mitochondria of BY4741-control, BY4741-EfLPA and BY4741-mEfLPA cells were extracted. The expression of EfLPA carrying 6xHis tag in mitochondria was confirmed by western blot analysis. (C) Lipoic acid production in vivo. Lipoic acid was extracted and quantified from BY4741-control, BY4741-EfLPA and BY4741-mEfLPA cells by LC-MS/MS analysis.

FIG. 7A-B shows production of lipoic acid using different engineered strains. (A) The overall pathway engineering for lipoic acid production. Dotted box represents the cofactor regeneration reaction catalyzed by Sam2p. (B) The comparison of total lipoic acid produced by the expression of different enzymes. “+” and “-” indicate presence and absence of the respective modifications. Data shown are the mean±SD of three biological replicates.

### DETAILED DESCRIPTION OF THE INVENTION

Bibliographic references mentioned in the present specification are for convenience listed in the form of a list of references and added at the end of the examples. The whole content of such bibliographic references is herein incorporated by reference. Any discussion about prior art is not an admission that the prior art is part of the common general knowledge in the field of the invention.

### Definitions

Certain terms employed in the specification, examples and appended claims are collected here for convenience.

It must be noted that as used herein and in the appended claims, the singular forms “a”, “an”, and “the” include plural reference unless the context clearly dictates otherwise.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

The terms “nucleotide”, “nucleic acid” or “nucleic acid sequence”, as used herein, refer to an oligonucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material.

As used herein, the term “operably linked” means that the components to which the term is applied are in a relationship that allows them to carry out their inherent functions under suitable conditions. For example, a control sequence which is “operably linked” to a protein coding sequence is ligated thereto, so that expression of the protein coding sequence is achieved under conditions compatible with the transcriptional activity of the control sequences. By way of an example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

As used herein, the terms “polypeptide”, “peptide” or “protein” refer to one or more chains of amino acids, wherein each chain comprises amino acids covalently linked by peptide bonds, and wherein said polypeptide or peptide can comprise a plurality of chains noncovalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. A “polypeptide”, “peptide” or “protein” can comprise one (termed “a monomer”) or a plurality (termed “a multimer”) of amino acid chains.

Media suitable for lipoic acid biosynthesis include LB broth, YPD, 2YT, and any other suitable culture media. The culture medium may include antibiotics such as ampicillin, kanamycin, chloramphenicol, Isopropyl p-D-1-galactopyranoside (IPTG), and L-arabinose. A person skilled in the art would know appropriate concentrations for each component.

A vector can include one or more catalytic enzyme nucleic acid(s) in a form suitable for expression of the nucleic acid(s) in a host cell. Preferably the recombinant expression vector includes one or more regulatory sequences operatively linked to the nucleic acid sequence(s) to be expressed. The term “regulatory sequence” includes promoters, enhancers, ribosome binding sites and/or IRES elements, and other expression control elements (e.g., polyadenylation signals). The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., catalytic enzyme proteins).

The recombinant expression vectors of the invention can be designed for expression of catalytic enzyme proteins in prokaryotic or eukaryotic cells, more particularly prokaryotic cells. For example, polypeptides of the invention can be expressed in bacteria (e.g., cyanobacteria) or yeast cells. Suitable host cells are discussed further in Goeddel, (1990) Gene Expression Technology: Methods in Enzymology 185, Academic Press, San Diego, Calif.Having now generally described the invention, the same will be more readily understood through reference to the following examples which are provided by way of illustration, and are not intended to be limiting of the present invention.

A person skilled in the art will appreciate that the present invention may be practiced without undue experimentation according to the methods given herein. The methods, techniques and chemicals are as described in the references given or from protocols in standard biotechnology and molecular biology textbooks.

### EXAMPLES

### Example 1

### Materials and Methods

### Strains and Media

E. coli TOP10 (Invitrogen) and Luria-Bertani (Becton, Dickinson and Company) were used for cloning experiments unless otherwise stated. 100 mg/L ampicillin was used for selection of positive colonies where applicable. The yeast strain S. cerevisiae BY4741 (ATCC) was used for genetic engineering for lipoic acid production.

S. cerevisiae BY4741 wild-type and mutant strains were cultured in rich medium YPD/YPGR (1% yeast extract, 2% peptone, and 2% D-glucose or 2% galactose with 1% raffinose), synthetic minimal medium lacking uracil SC-U (0.67% yeast nitrogen base, 0.192% uracil dropout and 2% D-glucose), medium lacking lysine SC-L (0.67% yeast nitrogen base, 0.18% lysine dropout and 2% D-glucose), medium lacking leucine SC-LE (0.67% yeast nitrogen base, 0.16% leucine dropout and 2% D-glucose), or medium lacking both leucine and uracil SC-LU (0.67% yeast nitrogen base, 0.154% leucine and uracil dropout, and 2% D-glucose). 2% agar was supplemented for making solid media. Yeast growth media components were purchased from Sigma-Aldrich, MP Biomedicals and BD (Becton, Dickinson and Company). 5-Fluoroorotic acid (5-FOA, Fermentas) or geneticin (G418, PAA Laboratories) was used for selection. Cysteine (0.2 mg/mL) and ferrous sulfate (0.2 mg/mL) (Sigma-Aldrich) were supplemented into growth culture where necessary. Yeast cells were cultivated at 30° C. in flasks and shaken at 225 rpm.

### Plasmid Construction and Gene Integration

EfLPA gene (GenBank Accession No. AY735444) was codon-optimized for S. cerevisiae and synthesized by Integrated DNA Technologies. EfLPA genes with and without mitochondrial targeting peptide (MTP) sequence were ligated between P_{GAL1} promoter and T_{CYC1} terminator, which were amplified from the S. cerevisiae genomic DNA. EfLPA expression cassettes with and without MTP were inserted to the vector pRS41K (Euroscarf), resulting in plasmids pRS41K-P_{GAL1}-mEfLPA-T_{CYC1} and pRS41K-P_{GAL1}-EfLPA-T_{CYC1}, respectively. The plasmids pRS41K-P_{GAL1}-mEGFP-T_{CYC1} and pRS41K-P_{GAL1}-EGFP-T_{CYC1} were similarly constructed for EGFP with and without MTP, respectively. The constructed recombinant plasmids are listed in Table 1. The list of primers used was shown in Table 2.

TABLE 1
Strains and plasmids used in this study

| Strains or plasmids | Description | Source |
|---|---|---|
| Strains | | |
| E. coli Top10 | F⁻ mcrA Δ(mrr-hsdRMS-mcrBC) φ80lacZΔM15 | 1 |
| | ΔlacX74 recA1 araD139 Δ(ara-leu)7697 galU galK | |
| | rpsL(Str^{R}) endA1 nupG | |
| S. cerevisiae | | |
| BY4741 | MATa his3Δ1 leu2Δ0 met15Δ0 ura3Δ0 | 2 |
| BY4741-GCV3 | BY4741 with P_{TEF1}-GCV3-T_{CYC1} (lys2 site) | 3 |
| BY4741-LAT1 | BY4741 with P_{TEF1}-LAT1-T_{ADH1} (lys2 site) | 3 |
| BY4741-KGD2 | BY4741 with P_{TEF1}-KGD2-T_{KGD2} (lys2 site) | 3 |
| BY4741-control | BY4741 with plasmid pRS41K | 3 |
| BY4741-EfLPA | BY4741 with plasmid pRS41K-P_{GAL1}-EfLPA-T_{CYC1} | 3 |
| BY4741-mEfLPA | BY4741 with plasmid pRS41K-P_{GAL1}-mEfLPA-T_{CYC1} | 3 |
| BY4741-EGFP | BY4741 with plasmid pRS41K-P_{GAL1}-EGFP-T_{CYC1} | 3 |
| BY4741-mEGFP | BY4741 with plasmid pRS41K-P_{GAL1}-mEGFP-T_{CYC1} | 3 |
| BY4741-GCV3- | BY4741 with P_{TEF1}-GCV3-T_{CYC1} (lys2 site) and plasmid | 3 |
| mEfLPA | pRS41K--P_{GAL1}-mEfLPA-T_{CYC1} | |
| BY4741-GCV3- | BY4741 with P_{TEF1}-GCV3-T_{CYC1} (lys2 site), P_{TEF1}-LIP2- | 3 |
| LIP2-LIP5--mEfLPA | T_{LIP2} (CS6 site), P_{PGI1}-LIP5-T_{LIP5} (CS6 site) and | |
| | plasmid pRS41K-P_{GAL1}-mEfLPA-T_{CYC1} | |
| BY4741-GCV3- | BY4741 with P_{TEF1}-GCV3-T_{CYC1} (lys2 site), P_{TEF1}-LIP2-T_{LIP2} | 3 |
| LIP2-LIP5--mSAM1- | (CS6 site), P_{PGI1}-LIP5-T_{LIP5} (CS6 site), P_{ADH1}-mSAM1-T_{SAM1} | |
| mEfLPA | (CS8) and plasmid pRS41K--P_{GAL1}-mEfLPA-T_{CYC1} | |
| BY4741-GCV3- | BY4741 with P_{TEF1}-GCV3-T_{CYC1} (lys2 site), P_{TEF1}-LIP2- | 3 |
| LIP2-LIP5--mSAM2- | T_{LIP2} (CS6 site), P_{PGI1}-LIP5-T_{LIP5} (CS6 site), P_{ADH1}- | |
| mEfLPA | mSAM2-T_{SAM2} (CS8) and plasmid pRS41K-P_{GAL1}- | |
| | mEfLPA-T_{CYC1} | |
| Plasmids | | |
| pIS385 | AmpR, URA3 | 4 |
| pRS41K | ARS/CEN origin, kanMX | 4 |
| pRS41K-P_{GAL1}- | pRS41K carrying EfLPA under P_{GAL1} control | 3 |
| EfLPA-T_{CYC1} | | |
| pRS41K-P_{GAL1}- | pRS41K carrying MTP-EfLPA under P_{GAL1} control | 3 |
| mEfLPA-T_{CYC1} | | |
| pRS41K-P_{GAL1}- | pRS41K carrying EGFP under P_{GAL1} control | 3 |
| EGFP-T_{CYC1} | | |
| pRS41K-P_{GAL1}- | pRS41K carrying MTP-EGFP under P_{GAL1} control | 3 |
| mEGFP-T_{CYC1} | | |

1. Invitrogen;
2. ATCC;
3. This study;
4. Euroscarf

TABLE 2
Primers used in this study. Restriction sites are in bold.

| | | SEQ |
|---|---|---|
| Primers | Primer Sequence 5’-3’ | ID NO |
| P_{GAL1}-F | AAACGAGCTCAGTACGGATTAGAAGCC | 13 |
| P_{GAL1}-R | TTTTTAGGGTTTTTTCTCCTTGACGTT | 14 |
| T_{CYC1}-F | ATCCGCTCTAACCGAAAAGG | 15 |
| T_{CYC1}-R | AAACGAGCTCCTTCGAGCGTCCCAAAACC | 16 |
| EfLPA-F | CGTCAAGGAGAAAAAACCCTAAAAAATGCTAGCCCAAGAA | 17 |
| mEfLPA- | CGTCAAGGAGAAAAAACCCTAAAAAATGCTTTCACTACGTCAATCT | 18 |
| F | ATAAGATTTTTCAAGCCAGCCACAAGAACTTTGTGTAGCTCTAGAT | |
| | ATCTGCTTCAGCAAAAACCCATGCTAGCCCAAGAA | |
| EfLPA-R | CTAACTCCTTCCTTTTCGGTTAGAGCGGATTCATTAATGGTGATGG | 19 |
| | TGATGATGCTTACGGGTCTTTCTAATGTAGA | |
| EGFP-F | CGTCAAGGAGAAAAAACCCTAAAAAATGTCTAAAGGTGAA | 20 |
| mEGFP- | CGTCAAGGAGAAAAAACCCTAAAAAATGCTTTCACTACGTCAATCT | 21 |
| F | ATAAGATTTTTCAAGCCAGCCACAAGAACTTTGTGTAGCTCTAGAT | |
| | ATCTGCTTCAGCAAAAACCCATGTCTAAAGGTGAA | |
| EGFP-R | CTAACTCCTTCCTTTTCGGTTAGAGCGGATTCATTAATGGTGATGG | 22 |
| | TGATGATGTTTGTACAATTCATC | |
| P_{TEF1}-F | ACCGCTCGAGCATAGCTTCAAAATGTTTCTACTCCTTT | 23 |
| P_{TEF1}-R | TTGTAATTAAAACTTAGATTAGATTGC | 24 |
| GCV3-F | GCAATCTAATCTAAGTTTTAATTACAAATGTTACGCACTACTAGACT | 25 |
| | ATGG | |
| GCV3-R | CTAACTCCTTCCTTTTCGGTTAGAGCGGATTCATTAATGGTGATGG | 26 |
| | TGATGATGGTCATCATGAACCAGTGT | |
| KGD2-F | GCAATCTAATCTAAGTTTTAATTACAAATGCTTTCCAGAGCGACG | 27 |
| KGD2-R | ATCAGATTGGTATGGGCTGCAAATTTCAAATCATTAATGGTGATGG | 28 |
| | TGATGATGCCATAACAACATTTTTCTAG | |
| T_{KGD2}-F | TTTGAAATTTGCAGCCCATAC | 29 |
| T_{KGD2}-R | ATTCGAGCTCATGTGGAAATCAAAAGAATATTAGTTGAT | 30 |
| LAT1-F | GCAATCTAATCTAAGTTTTAATTACAAATGTCTGCCTTTGTCAGGGT | 31 |
| | G | |
| LAT1-R | TAATAAAAATCATAAATCATAAGAAATTCGTCATTAATGGTGATGGT | 32 |
| | GATGATGCAATAGCATTTCCAAAGGAT | |
| T_{ADH1}-F | CGAATTTCTTATGATTTATGATTTTTA | 33 |
| T_{ADH1}-R | ACGCGGATCCGAGCGACCTCATGCTATACCT | 34 |
| LIP2- | AACCTCGAGGAGAAGTTTTTTTACCCCTCTCCACAGATCCTCGAGC | 35 |
| LIP5- | ATAGCTTCAAAATGTTTCTAC | |
| CS6-F | | |
| LIP2- | TAATTAGGTAGACCGGGTAGATTTTTCCGTAACCTTGGTGTCGAGC | 36 |
| LIP5- | TCACGCATTTTTTTCTTTTGC | |
| CS6-R | | |
| SAM1/2- | CAAAATTACCTACGGTAATTAGTGAAAGGCCAAAATCTAATGTTAC | 37 |
| CS8-F | AATAGTATACTAGAAGAATGAGCCAAG | |
| SAM1- | GACCGTTCCCTTGTGTTGTACCAGTGGTAGGGTTCTTCTCGGTAG | 38 |
| CS8-R | CTTCTATAAGATAAAGTTTGGTTTGTTGATC | |
| SAM2- | GACCGTTCCCTTGTGTTGTACCAGTGGTAGGGTTCTTCTCGGTAG | 39 |
| CS8-R | CTTCTCCTCAAAGACATTCTATATTTCAACC | |

Chromosomal integration of the expression cassettes P_{TEF1}-GCV3-T_{CYC1}, P_{TEF1}-KGD2-T_{KGD2} and P_{TEF1}-LAT1-T_{ADH1} into the LYS2 site were conducted based on the method previously described by Sadowski et al. (Sadowski et al., Yeast 24: 447-455 (2007)), where the integrative vector pIS385 (Euroscarf) containing URA3 selectable marker was used for integration. In addition, the cassettes P_{TEF1}-LIP2-T_{LIP2} and P_{PGI1}-LIP5-T_{LIP5} were integrated into intergenic site CS6 while P_{ADH1}-mSAM1-T_{SAM1} and P_{ADH1}-mSAM2-T_{SAM2} were integrated into intergenic site CS8 (Xia et al., ACS Synthetic Biology 6: 276-283 (2017)) based on Clustered Regularly Interspaced Short Palindromic Repeats (CRISPR) and CRISPR-associated (Cas) system previously established (DiCarlo et al., Nucleic Acids Research 41: 4336-4343 (2013)). To clone GCV3, LAT1, KGD2, LIP2, LIP5, SAM1 and SAM2, genomic DNA of S. cerevisiae was used as the PCR template. All proteins abovementioned were located to the mitochondria through its native MTP (for Gcv3p, Lat1p and Kgd2p) or MTP from yeast cytochrome c oxidase subunit IV (COX4) (for mEfLPA, mSam1p and mSam2p) (Maarse et al., The EMBO Journal 3: 2831-2837 (1984)). Hexa-histidine tag was added to either the C- or N-terminus of these proteins for expression analysis. Oligonucleotide primers used are listed in Table 2.

### Detection of Lipoylated and Octanoylated Proteins

Cells were pre-cultured in 5 ml yeast extract peptone dextrose (YPD) medium overnight and then diluted in 100 ml YPD medium using 500 ml flask to achieve an initial OD₆₀₀ of 0.4. After growth for 18 h, cells were harvested by centrifugation. Cell pellets were re-suspended in 25 ml lysis buffer (0.3 M NaCl, 50 mM sodium phosphate, pH 6.5). Cells were lysed with a high-pressure homogenizer (EmulsiFlex-C3, AVESTIN, Inc.) at 25000 psi. The soluble cell lysate was collected by centrifugation and mixed an equal volume of 8 M Guanidine hydrochloride. 300 μl final products were injected into Agilent 1260 Infinity binary HPLC (Agilent). The proteins were resolved with an mRP-C18 High-Recovery Protein column (Agilent) at a solvent flow rate of 1.5 ml/min and column temperature of 80° C. The mobile phases A and B were 0.1% trifluoroacetic acid/water and 0.1% trifluoroacetic acid/acetonitrile respectively. The proteins were eluted with the following gradient: 0-1 min (10%-30% B), 1-12 min (30%-50% B), 12-13 min (50%-80% B), 13-14 min (80% B), 14-15 min (80%-10% B) and 15-17 min (10% B). Protein collection started from 1 min and 12 successive 1-min fractions were collected. The proteins were dried overnight in a Speedvac concentrator (Thermo Fisher Scientific). Each fraction of proteins was re-suspended with 50 μl 0.5 M triethylammonium bicarbonate with 1 μg Glu-C(Promega). The mixture was incubated overnight.

7 μl digested peptides was loaded into Agilent 1260 infinity HPLC-Chip/MS System (Agilent) equipped with a PortID-Chip-43 (II) column (Agilent). A linear gradient of acetonitrile was used to elute the peptides from the HPLC-Chip system at a consistent flow rate of 0.35 μl/min. For LC separation, 0.2% formic acid/water (mobile phase A) and 0.2% formic acid/acetonitrile (mobile phase B) were used. The samples were eluted with the following gradient through a nano pump: 0-1 min (7%-10% B), 1-35 min (10%-30% B), 35-37 min (30%-80% B), 37-38 min (80% B), 38-40 min (80%-7% B) and 40-43 min (7% B). The eluted samples were directly infused into a mass spectrometer for detection. The mass spectra were scanned in the range of 100-1600 m/z with a scan rate of 3 spectra per second. The MS/MS scan range is 80-2000 m/z with a scan rate of 4 spectra per second. Mass data was collected in positive ion mode at a fragmentor voltage of 175 V and skimmer voltage of 65 V.

### Peptide Post-Translational Modification (PTM) Analysis

The SPIDER feature of PEAKS 8 software (Bioinformatics Solutions Inc., Waterloo, Canada) (Zhang et al., Molecular & Cellular Proteomics: MCP 11: M111.010587 (2012)) was used to identify the peptides with PTMs based on mass difference. The yeast peptides were searched with the following search parameters. The precursor mass error tolerance was 100 ppm (part-per-million) while the fragment mass error tolerance was 0.1 Da. The fixed PTM was carbamidomethylation(C) (+57.02) and variable PTMs were lipoyl (K) (+188.03), octanoyl (TS) (+126.10), oxidation (M) (+15.99) and oxidation (HW) (+15.99). The peptide and protein identification reliability score (−10lgP, where P is the probability of identification) was set at a threshold of 15 and 20 respectively, corresponding to confident identifications.

### The Database Used was UniProtKB/Swiss-Prot.

Protein modeling for structure visualization SWISS-MODEL (Waterhouse et al., Nucleic Acids Research 46: W296-W303 (2018)) was used to build the 3D structure models of Gcv3p, Kgd2p and Lat1p proteins from their amino acid sequences using homology modelling techniques. The structures were predicted based on templates available in the SWISS-MODEL template library (SMTL) which aggregates information of experimental structures from Protein Data Bank (PDB). PyMOL Molecular Graphics System (Schrödinger, Inc., New York, USA) (Schrödinger, “The PyMOL Molecular Graphics System, Version 1.8” (2015)) was used to observe the structures.

Template homologue proteins with 41%, 37% and 48% sequence identity were used for modelling of Gcv3p, Kgd2p and Lat1p, respectively. The template protein for Gcv3p is glycine cleavage system protein H from Mycobacterium tuberculosis (PDB chain id: 3hgb.1.A), while for Kgd2p and Lat1p, only the N-termini (lipoyl domains) were modelled due to the lack of templates with crystal structure of full length. The template for the N-terminus (lipoyl domain) of Kgd2p is the lipoyl domain of E2 component of 2-oxoglutarate dehydrogenase complex in Azotobacter vinelandii (PDB chain id: 1ghj.1.A). The N-terminus of Lat1p (lipoyl domain) was modelled using the dihydrolipoyllysine-residue acetyltransferase component of the pyruvate dehydrogenase complex in Homo sapiens (PDB chain id: 1y8n.1.B).

### Protein Overexpression and Purification

Cells were pre-cultured in 5 ml medium overnight and then diluted in 50 ml induction medium using 200 ml flask to achieve an initial OD₆₀₀ of 0.4. After overnight cell growth, the yeast cells were harvested by centrifugation. The cell pellets were re-suspended in lysis buffer (0.5 M NaCl, 20 mM sodium phosphate, 20 mM imidazole, pH 6.8) and lysed with a high-pressure homogenizer (EmulsiFlex-C3, AVESTIN, Inc.) at 25000 psi. After centrifugation, the insoluble protein and cell debris were separated from the soluble protein. To check protein expression, the soluble protein was boiled with Laemmli sample buffer (Bio-Rad) and separated on an SDS-polyacrylamide gel. The proteins in the gels were transferred onto western blotting membrane and using HRP conjugated anti-6x His-tag antibody (ThermoFisher Scientific) as described previously (Chen et al., Biotechnology for Biofuels 6: 21 (2013)). To detect protein expressed in the mitochondria, mitochondrial proteins were extracted using yeast mitochondria isolation kit (Biovision). The extracted proteins will be boiled with Laemmli sample buffer and detected through western blotting as described.

To purify the proteins, the soluble proteins were incubated with Nickel-IMAC resin (GE Healthcare) overnight for protein binding. After protein binding and washing, the His-tagged proteins were eluted with elution buffer (0.5 M NaCl, 20 mM sodium phosphate, 300 mM imidazole, pH 6.8). Protein concentrator (Thermo Scientific) was used to exchange the elution buffer with PBS buffer for downstream protein activity test.

### Free Lipoic Acid Detection

The extraction and detection of free lipoic acid using the LC-MS/MS method developed by Chng et al. (Chng et al., Journal of Pharmaceutical and Biomedical Analysis 51: 754-757 (2010)) with modifications. Equal volume of acetonitrile was added to the supernatant of cell culture or lysate. The mixture was vortex-mixed for 2 min. After cooling at −30° C. for 30 min, the upper phase containing lipoic acid was transferred to a clean tube for evaporation to dryness. The residue was reconstituted with 200 μl of 50% acetonitrile in water. The extracted lipoic acid sample was injected into an LC-MS/MS system (Agilent 1290 liquid chromatograph and Agilent 6550 iFunnel Q-TOF) in negative mode. Chromatographic separation was achieved with an Agilent Eclipse Plus C18 column (2.1×100 mm, 1.8 μm, Agilent) at a flow rate of 0.7 ml/min by gradient solution at 0-5.8 min (80%-68% A), 5.8-6.5 min (68%-15% A) and 6.5-7 min (15%-95% A). Mobile phase A is 0.1% acetic acid (pH 4 adjusted with ammonia hydroxide solution) and mobile phase B is acetonitrile. Nebulizer was set at 40 psig, while sheath gas flow rate is 11 l/min. The optimized collision energy for lipoic acid is 8 eV. Quantification was achieved by using 2-propylvaleric Acid (Tokyo Chemical Industry Co., Ltd.) as an internal standard.

Gas chromatography-mass spectrometry (GC-MS) was also used to confirm the identity of lipoic acid. Briefly, HPLC grade ethyl acetate (Sigma) was added to either the supernatant of the cell culture or lysate to extract lipoic acid. The mixture was separated into two phases by centrifugation. The upper phase containing lipoic acid was mixed with N,O-bis(trimethylsilyl)trifluoroacetamide (BSTFA) containing 1% trimethylchlorosilane at a ratio 4:1. The derivatized lipoic acid was analyzed using GC-MS under the following conditions. An HP-5 ms column (30 m by 0.25 mm; 0.25 μm film; Agilent) was used with a helium flow rate set to 1 ml/min. Injections of 1 μl were carried out under splitless injection condition with the inlet set to 250° C. The GC temperature profile was as follows: an initial temperature of 45° C. was maintained for 2 min, followed by ramping to 280° C. at a rate of 10° C./min, where the temperature was held for 3.5 min. The mass spectrometer detector was scanned from 30 to 800 amu in the electron ionization (EI) mode. To aid peak identification, authentic lipoic acid (Sigma) standard was used as reference.

### Fluorescence Microscopy

S. cerevisiae BY4741 cells carrying the plasmids pRS41K-P_{GAL1}-EGFP-T_{CYC1} and pRS41K-P_{GAL1}-mEGFP-T_{CYC1} were grown to early logarithmic phase in induction medium (YPGR with 200 mg/L G418). The cells were harvested and mounted on a poly-L-lysine-coated glass slide. EGFP fluorescence was visualized with a fluorescent microscope (Leica DMi8).

### Example 2

Proteomic analysis and characterization of lipoylated proteins as substrates for free lipoic acid biosynthesis

To engineer the yeast for free lipoic acid biosynthesis, we first aimed to evaluate the availability of the various forms of lipoate-bound proteins and understand their formation process. We hypothesized that this would facilitate our selection of a suitable lipoylated protein as substrate for subsequent enzymatically cleavage by EfLPA at the amide linkage to release free lipoic acid. Lipoic acid exists covalently bound to proteins via an amide linkage in S. cerevisiae. It was hypothesized that its biosynthesis begins with the transfer of an octanoyl moiety from octanoyl-ACP to the apo form of lipoate-dependent proteins, followed by modification of the octanoyl moiety by insertion of two sulfur atoms (Schonauer et al., Journal of Biological Chemistry 284: 23234-23242 (2009)). As lipoic acid is mainly bound to three proteins, namely Gcv3p, Lat1p and Kgd2p, we sought to focus our analysis on these proteins through LC-MS/MS to better our understanding of the protein lipoylation mechanism.

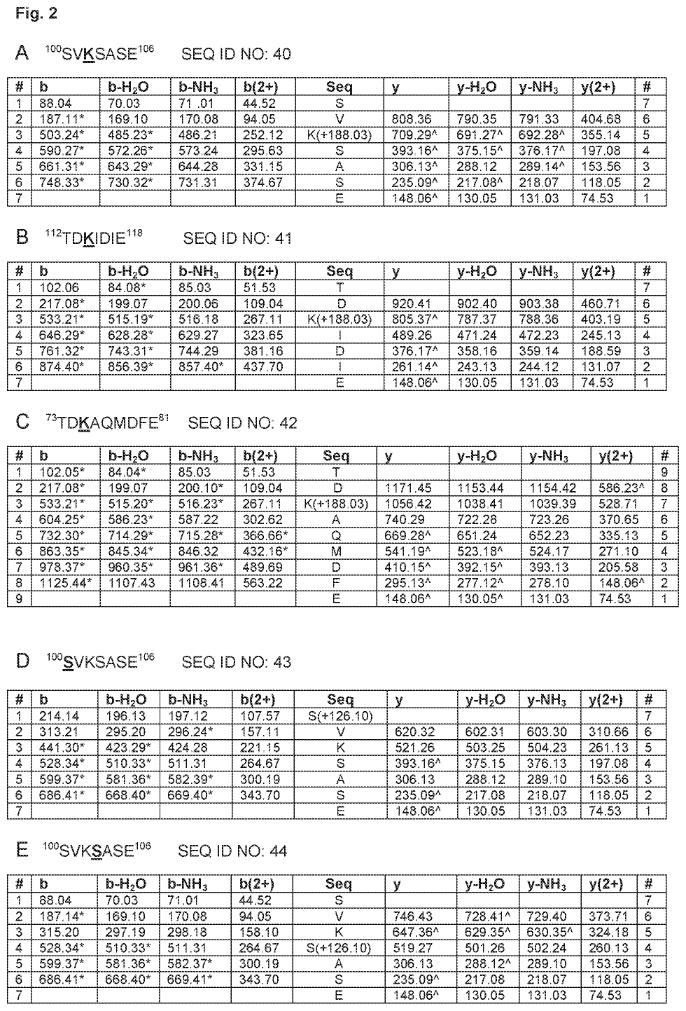

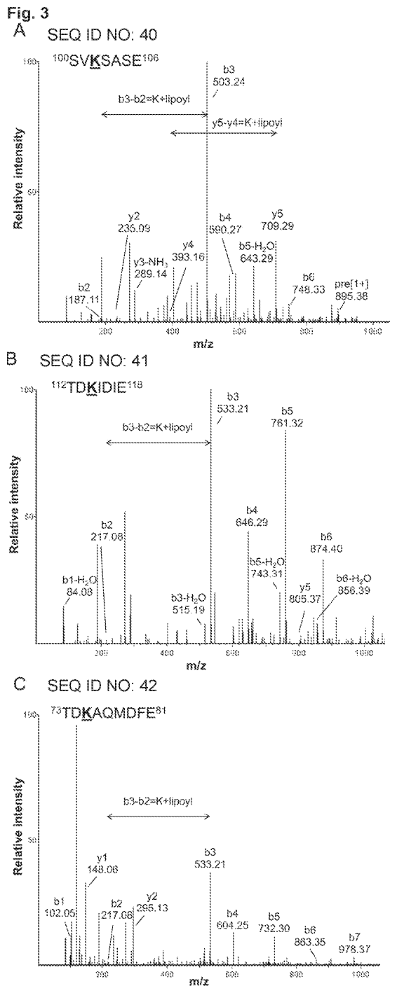

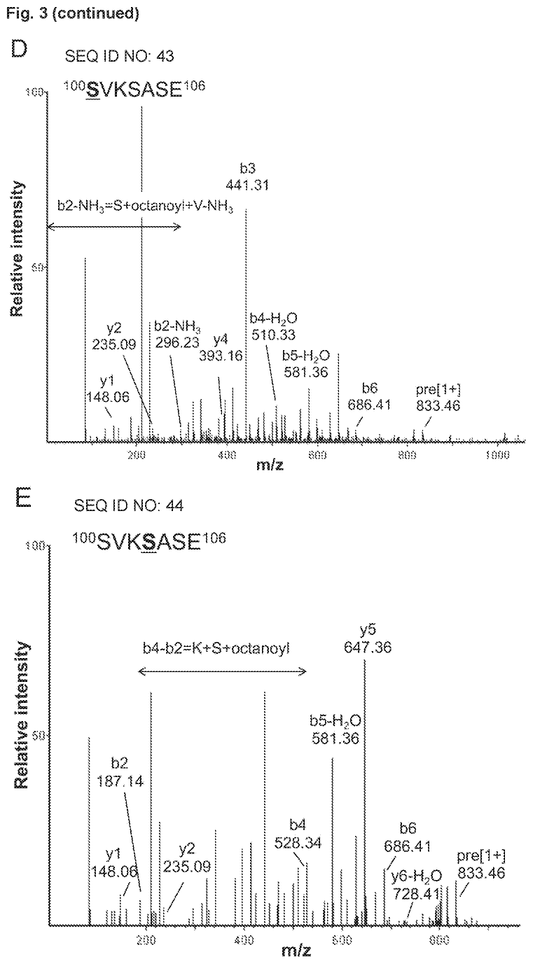

To investigate the lipoylation of Gcv3p, Lat1p and Kgd2p, we extracted the total protein from S. cerevisiae and separated the proteins into 12 fractions by HPLC with reverse phase column to reduce the complexity of our protein samples. Instead of using trypsin and chymotrypsin reported previously to generate long peptide fragments (Gey et al., PLoS one 9:e103956 (2014)), in this study, each protein sample was digested with Glu-C leading to shorter peptides which gives better precision. The digested peptide mixtures were analyzed by LC-MS/MS. In total, 2,713 peptides were identified based on their m/z value and MS/MS spectra. As shown in FIG. 3A, a singly charged peptide with m/z 895.3918 was detected. This fragment corresponds to the ¹⁰⁰SVKSASE¹⁰⁶ (SEQ ID NO: 40) sequence from Gcv3p carrying a lipoic acid modification at K¹⁰² (lysine¹⁰²). Similarly, a singly charged peptide with m/z 1021.4584 revealed the presence of the sequence¹¹²TDKIDIE¹¹⁸ (SEQ ID NO: 41) from Kgd2p with K¹¹⁴ modified by lipoic acid (FIG. 3B). A doubly charged lipoylated peptide with m/z 636.7529 detected as a precursor ion indicates that the sequence⁷³TDKAQMDFE⁸¹ (SEQ ID NO: 42) from Lat1p was also modified with lipoic acid at K⁷⁵ (FIG. 3C). Therefore, we concluded from our data that Gcv3p, Kgd2p and Lat1p were lipoylated at positions K¹⁰² K¹¹⁴ and K⁷⁵, respectively, in wild-type cell BY4741. The detailed calculations are shown in FIG. 2.

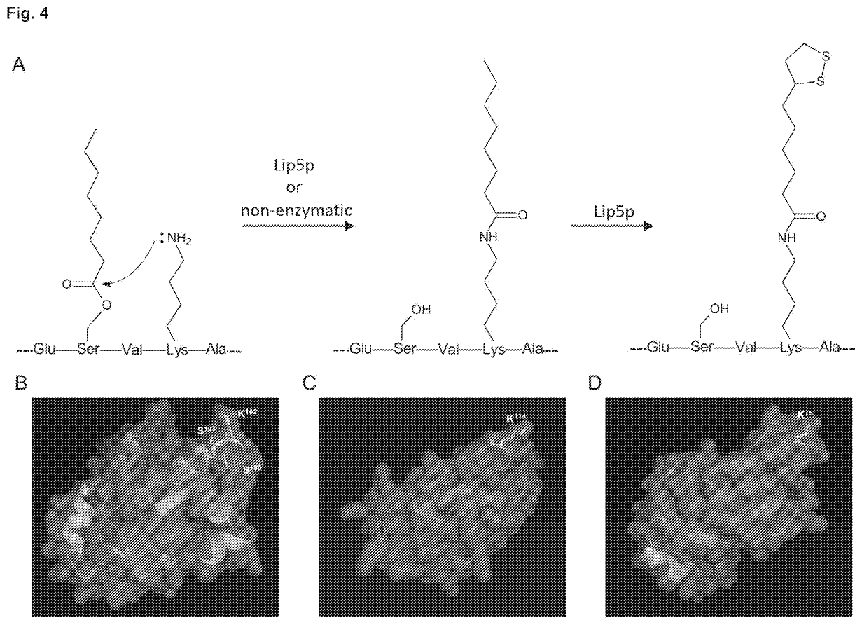

In addition to lipoylated peptides, we also observed octanoylated peptides in Gcv3p that likely originated from precursors of lipoate-proteins. Detection of two singly charged peptides with m/z 833.4583 and 833.4628 indicates single octanoyl modification of the Gcv3p fragment at the S¹⁰⁰ (serine¹⁰⁰) (¹⁰⁰SVKSASE¹⁰⁶; SEQ ID NO: 43) or S¹⁰³ position (¹⁰⁰SVKSASE¹⁰⁶; SEQ ID NO: 44), respectively (FIGS. 3D and 3E). This suggests that, unexpectedly, binding of lipoate and octanoate does not occur on the same residue but instead takes place on lysine and proximal serine residues, respectively. These data provide the first MS-based evidence of octanoylation of Gcv3p protein at serine residues in the vicinity of the lipoate-modified lysine residue, inferring that Gcv3p is loaded with octanoate at S¹⁰⁰ or S¹⁰³ to serve as precursors prior to the formation of lipoate-Gcv3p with lipoate-modified K¹⁰². Therefore, instead of direct octanoylation of lysine followed by addition of sulfur atoms to the octyl carbon chain, we propose that lipoyl-Gcv3p is formed through the following three steps: (i) esterification of the serine (S¹⁰⁰ or S¹⁰³) side chain with an octanoyl functional group, (ii) amidation of the lysine (K¹⁰²) side chain by acyl transfer of the octanoyl moiety from S¹⁰⁰ or S¹⁰³ and (iii) insertion of sulfur atoms to the octanoyl moiety by the lipoyl synthase Lip5p (FIG. 4A). Interestingly, no octanoylated peptides derived from Kgd2p and Lat1p were detected. One possibility is that octanoylated Kgd2p and Lat1p proteins can be intermediately converted to lipoate-modified protein after they were generated. Alternatively, lipoylation of Kgd2p and Lat1p may occur via amido-transfer from lipoate-Gcv3p, since Gcv3p and Lip3p are essential for forming lipoate-modified Kgd2p and Lat1p, and Lip3p has been suggested to be a possible amidotransferase (Schonauer et al., Journal of Biological Chemistry 284: 23234-23242 (2009); Hiltunen et al., Biochmica et Biophysica Acta (BBA)-Bioenergetics 1797: 1195-1202 (2010)).

To elucidate the protein structural characteristics and visualize the locations of the octanoylation and lipolylation sites, we predicted the structures of Gcv3p, Kgd2p and Lat1p by homology modeling (FIGS. 4B, 4C and 4D). All the residues for modification, namely K¹⁰² S¹⁰⁰ and S¹⁰³ in Gcv3p, K¹¹⁴ in Kgd2p and K⁷⁵ in Lat1p, are positioned on p-turns, which are typically surface-exposed (Marcelino and Gierasch, Biopolymers 89: 380-391 (2008)). Hence, their corresponding octanoyl- and lipoyl-PTMs are present on the protein surface and accessible for enzymatic catalysis to be performed on these residues, i.e. the attachment of octanoic acid to serine residue by Lip2p/Lip3p, the insertion of sulfur atoms to the octanoylated lysine residue by Lip5p and the hydrolysis of the amide bond between the lipoic acid and the lysine residue by EfLPA. Overall, we identified the lysine residues where Gcv3p, Kgd2p and Lat1p are lipoylated in wild-type BY4741 strain, i.e. K¹⁰², K¹¹⁴ and K⁷⁵, respectively. The discovery of octanoylated serine residues in Gcv3p suggests a lipoylation mechanism whereby octanoylation of the lysine residue involves pre-loading of octanoyl moiety onto serine residues followed by acyl transfer to the lysine side chain. We have also established from the predicted protein structures of Gcv3p, Kgd2p and Lat1p that their lipoylated lysine residues are accessible to EfLPA for hydrolysis. Hence the activity of EfLPA on lipoylated Gcv3p, Kgd2p and Lat1p was subsequently characterized to determine the suitability of these lipoylated enzymes as substrates for EfLPA to produce free lipoic acid.

### Example 3

### In Vitro Characterization of EfLPA for Free Lipoic Acid Biosynthesis

Free lipoic acid is produced by enzymatic cleavage of the amide bond linking the lipoyl moiety to the lysine of lipoate-dependent proteins with a lipoamidase. EfLPA from E. faecalis was previously shown to release lipoic acid from lipoate-modified proteins in E. coli (Spalding and Prigge, PLoS one 4: e7392 (2009)). Lipoic acid is mainly bound to three proteins, namely Gcv3p, Lat1p and Kgd2p in yeast as demonstrated in FIG. 3, but whether EfLPA is functional towards these lipoylated yeast proteins has not been reported. Therefore, to engineer S. cerevisiae for free lipoic acid biosynthesis, we characterized the in vitro enzyme activity of EfLPA towards these lipoylated proteins. We hypothesized that through this in vitro investigation, we could identify a suitable substrate protein candidate that EfLPA is catalytically active on for subsequent overexpression to increase the availability of sites at which lipoic acid can be synthesized.

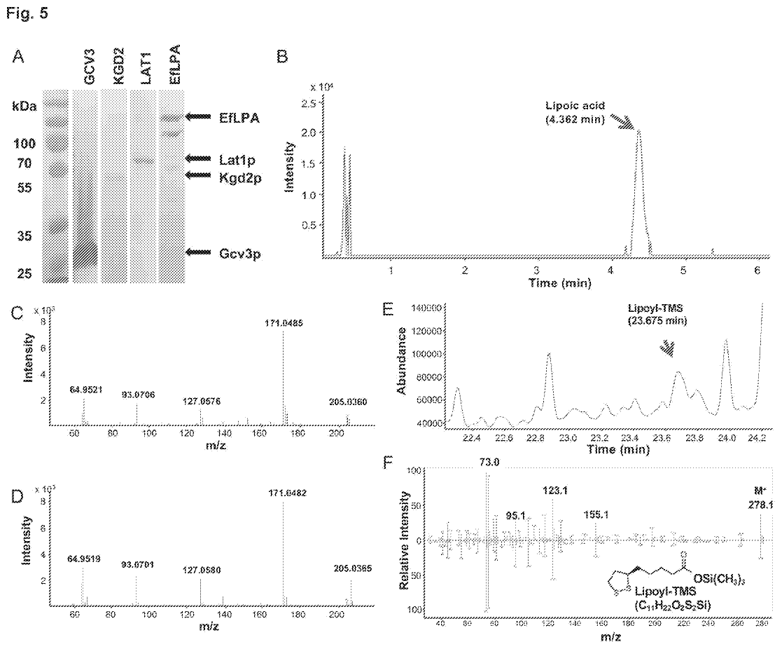

To test the catalytic activity of EfLPA towards lipoylated proteins from yeast, EfLPA with hexa-histidine tag was expressed under the strong galactose-inducible P_{GAL1} promoter from a low copy-number plasmid. Lipoate-bound proteins (i.e. Gcv3p, Kgd2p and Lat1p) fused with a hexa-histidine tag were expressed individually under the strong constitutive promoter P_{TEF1} from the genome. As shown in FIG. 5A, the expression of Gcv3p, Kgd2p, Lat1p and EfLPA in S. cerevisiae was confirmed by western blot. Gcv3p showed much higher protein expression than the other proteins, while Kgd2p showed the lowest protein expression. The reason for the low expression levels of Kgd2p and Lat1p is unclear but it has been shown that essential proteins have relatively shorter protein half-lives, which may be due to strict fidelity requirements and lower threshold to damage for essential proteins (Martin-Perez and Vill6n, Cell Systems 5: 283-294.e285 (2017)). Therefore, the low protein expression of Kgd2p and Lat1p may be due to fast protein turnover since both Kgd2p and Lat1p are involved in aerobic respiration, a central process in cellular metabolism (Schonauer et al., Journal of Biological Chemistry 284: 23234-23242 (2009)). Western blot analysis of EfLPA protein showed multiple bands, which is consistent with a previous report (Spalding and Prigge, PLoS one 4: e7392 (2009)).

To determine whether EfLPA possesses broad-range lipoamidase activity towards lipoylated proteins from yeast, purified Gcv3p, Kgd2p and Lat1p proteins were incubated with purified EfLPA individually at 37° C. for 2 h. The extracted products from the enzymatic reaction mixtures were analyzed by LC-MS/MS. No lipoic acid was detected in the control reaction mixture containing EfLPA, Gcv3p, Kgd2p or Lat1p only. Interestingly, no lipoic acid was observed in the reaction mixtures containing EfLPA with Kgd2p or Lat1p individually. Only the reaction of EfLPA with Gcv3p resulted in a peak with m/z 205.0360 (FIG. 5B) indicative of lipoic acid. Product ion scan of the abovementioned precursor ion m/z 205.0360 displayed clear and abundant product ions at m/z 64.9521, 93.0706, 127.0576 and 171.0485 (FIG. 5C), which is identical to the mass spectrum of a lipoic acid reference standard (FIG. 5D). The extracted product was additionally analyzed by GC-MS to further confirm the presence of lipoic acid. Analysis of the trimethylsilyl derivatized product showed a peak with a corresponding mass spectrum identical to that of the reference standard (FIGS. 5E and 4F). These results demonstrate that EfLPA has lipoamidase activity towards Gcv3p from yeast in vitro and can be potentially used as an amidohydrolase to release free lipoic acid from lipoate-modified proteins in yeast. It is unclear why no lipoic acid was generated by EfLPA from Kgd2p or Lat1p. Structure models of Gcv3p, Kgd2p and Lat1p show that all the modified residues, i.e. K¹⁰² S¹⁰⁰ and S¹⁰³ in Gcv3p, K¹¹⁴ in Kgd2p and K⁷⁵ in Lat1p, are present on p-turns exposed to the solvent on the protein surface, and hence inaccessibility of the lipoylation site is unlikely the reason for the lack of lipoamidase activity of EfLPA on Kgd2p and Lat1p. Other possibilities may be that (i) the protein expression levels of Lat1p and Kgd2p were too low (FIG. 5A), (ii) less lipoic acid moiety were attached to Lat1p and Kgd2p proteins compared with Gcv3p (Hermes and Cronan, Yeast 30: 415-427 (2013)) or (iii) the substrate specificity of EfLPA excludes both Lat1p and Kgd2p.

Taken together, the in vitro results show that Gcv3p, being a better substrate for EfLPA compared to Lat1p and Kgd2p, is the most suitable protein substrate out of the three candidates for subsequent pathway engineering to optimize free lipoic acid biosynthesis. Moreover, Gcv3p is a smaller protein than Kgd2p and Lat1p (19 kDa, 50 KDa and 52 kDa, respectively), and thus its overexpression utilizes less resource than the latter proteins. Furthermore, unlike the formation of lipoate-Gcv3p, lipoylation of Kgd2p and Lat1p requires an additional enzyme, i.e. Lip3p, which might reduce the efficiency of lipoylation and increase metabolic burden if LIP3 overexpression is additionally required. In summary, we established that EfLPA is functionally expressed in S. cerevisiae and has activity on Gcv3p, which we therefore selected as the preferred lipoylated protein substrate. These enzymes were employed for subsequent engineering of S. cerevisiae to overproduce free lipoic acid in vivo.

### Example 4

### Overexpression of EfLPA in the Mitochondria LED to Lipoic Acid Biosynthesis In Vivo

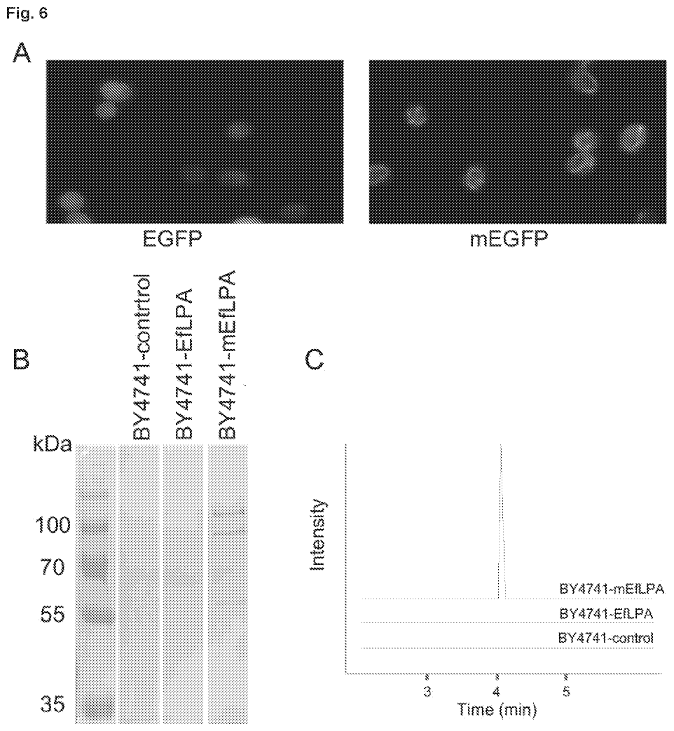

As mentioned, lipoic acid synthesis occurs in the mitochondria of yeast. To enable lipoic acid biosynthesis in vivo, EfLPA must be translocated to the mitochondria where it hydrolyzes lipoic acid from lipoylated protein substrates. To this end, a 29-amino-acid mitochondrial targeting peptide (MTP) from the yeast cytochrome c oxidase subunit IV (COX4) (Maarse et al., The EMBO Journal 3: 2831-2837 (1984)) was explored for translocating proteins to the mitochondria. As shown in FIG. 6A, EGFP fused with the MTP was localized in the mitochondria while EGFP without MTP was diffused in the cytosol. To localize EfLPA to mitochondria, EfLPA was fused with the characterized MTP. Mitochondrial proteins were extracted and analyzed by western blotting to determine mitochondrial translocation of EfLPA. Only the extracts from cells expressing MTP-EfLPA fusion protein (mEfLPA) showed a band corresponding to the protein whereas no bands were observed in the extracts from wild-type BY4741 with empty plasmid and cells expressing EfLPA without MTP, hence confirming translocation of EfLPA to the mitochondria when fused with MTP (FIG. 6B).

We evaluated the in vivo activity of the EfLPA in mitochondria by quantifying the lipoic acid concentrations in cell cultures grown for 3 d. We found that the wild-type BY4741 with empty plasmid and BY4741 expressing EfLPA without MTP produced no detectable lipoic acid, whilst the BY4741-mEfLPA strain expressing EfLPA in the mitochondria produced free lipoic acid at 10.1 μg/L (FIG. 6C). Thus, BY4741-mEfLPA constructed here is the first yeast strain reported with the ability to produce free lipoic acid in vivo and served as the base strain for further engineering to improve titer.

### Example 5

### Expression of Pathway Enzymes and Regeneration of Cofactor Improved Lipoic Acid Production

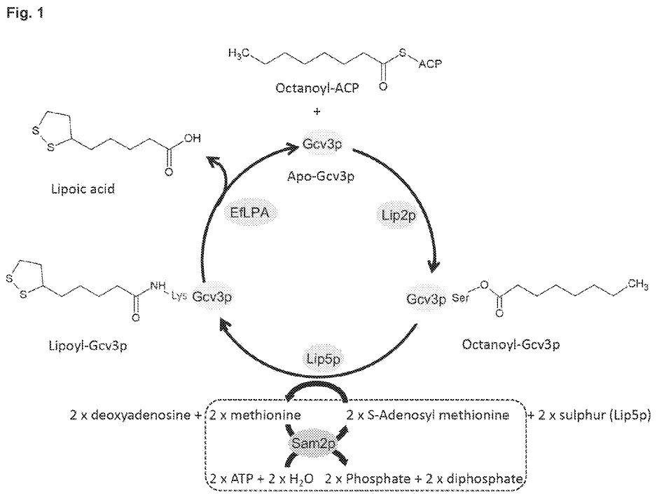

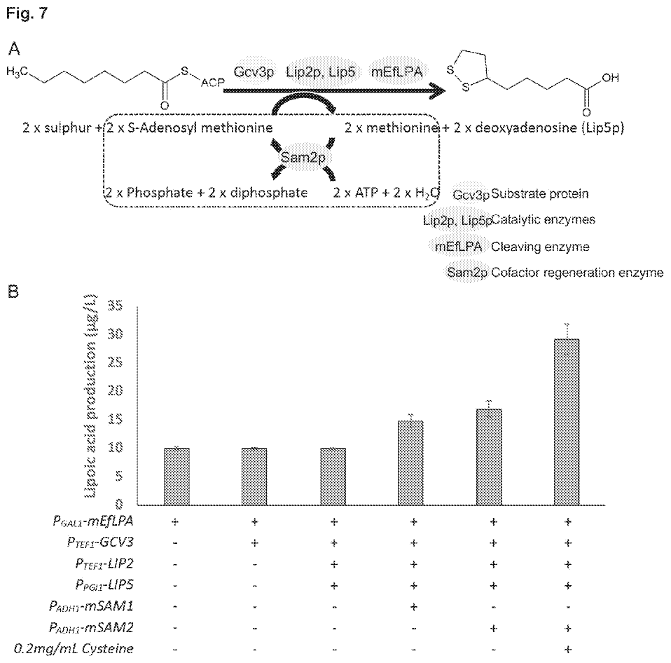

The overall genetic engineering for lipoic acid production in vivo is shown in FIG. 7A. As a first step to improve lipoic acid production, we attempted to increase the availability of lipoylation sites by overexpressing a suitable protein candidate such that more lipoylated proteins can form to serve as substrates for EfLPA hydrolysis. Specifically, as determined in section 3.2, GCV3p was selected to be the protein candidate for overexpression. To this end, we co-expressed GCV3 under P_{TEF1} from the genome along with mEfLPA, hence generating the strain BY4741-GCV3-mEfLPA. However, as shown in FIG. 7B, overexpression of GCV3p did not improve free lipoic acid production. This suggests that the bottleneck in free lipoic acid production from strain BY4741-mEfLPA is not the inadequacy of substrate protein, which can be recycled during free lipoic acid production, but possibly insufficient activity of the catalytic enzymes and/or cofactors required to synthesize the lipoyl moiety (FIG. 1).

The catalytic enzyme Lip2p, an octanoyltransferase, has been demonstrated to convert apo-Gcv3p to octanoyl-Gcv3p while another catalytic enzyme Lip5p, a lipoyl synthase, catalyzes the conversion of octanoyl-Gcv3p to lipoyl-Gcv3p (Hermes and Cronan, Yeast 30: 415-427 (2013)) (FIG. 1). Thus, to increase the level of lipoyl-Gcv3p, LIP2 was expressed under the strong P_{TEF1} promoter while LIP5 was expressed under the weak P_{PGI1}promoter (as expression of LIP5 under the strong P_{TEF1} promoter caused cell inviability). However, the resulting strain overexpressing GCV3, LIP2, LIP5 and mEfLPA showed similar lipoic acid production compared with cells expressing mEfLPA only (FIG. 7B), suggesting that the activities of Lip2p and Lip5p are not rate-limiting for lipoic acid production.

Another possible rate-limiting factor for lipoic acid production in yeast is the availability of cofactors, particularly S-adenosylmethionine (SAM), which is required for sulfurization of the octanoyl moiety. Homologous lipoyl synthase from E. coli uses radical SAM chemistry to perform the insertion of two sulfurs into the octanoyl moiety, a process that requires both the cofactor SAM and the iron-sulfur clusters in the lipoyl synthase (Cicchillo et al., Biochemistry 43: 6378-6386 (2004)). Radical intermediates are generated from SAM to abstract hydrogen atoms from C-6 and C-8 of the octanoyl moiety, allowing for subsequent sulfur insertion by a mechanism involving carbon-centered radicals. Iron-sulfur cluster in the lipoyl synthase provides an electron during the cleavage of SAM for radical generation and also may act as the source for sulfur atoms during lipoylation (Cicchillo and Booker, Journal of the American Chemical Society 127: 2860-2861 (2005)). Therefore, increasing the availability of SAM and functional iron-sulfur clusters may drive the formation of lipoyl moiety. In S. cerevisiae, SAM can be generated from methionine and ATP by the lipoyl synthases Sam1p and Sam2p (Marobbio et al., The EMBO Journal 22: 5975-5982 (2003); Dato et al., Microbial Cell Factories 13: 147 (2014)). To increase SAM availability by regeneration from methionine and ATP, SAM1 and SAM2 were fused with MTP for mitochondria translocation and overexpressed under the weak P_{ADH1} promoter. Overexpression of the mitochondrial mSAM1 or mSAM2 increased lipoic acid production to 14.8 μg/L and 17.0 μg/L, respectively (FIG. 7B), suggesting that SAM availability is a critical bottleneck in lipoic acid production. To form the iron-sulfur clusters in the lipoyl synthases, ferrous ions need to be imported from the medium and sulfur has to be released from cysteine through the iron-sulfur cluster assembly machinery (Lill et al., Biochimica et Biophysica Acta (BBA)—Molecular Cell Research 1763: 652-667 (2006)). Therefore, to further drive the synthesis of the lipoyl moiety, the cell culture of the highest lipoic acid producer, i.e. the strain overexpressing GCV3, LIP2, LIP5, mSAM2 and mEfLPA, was supplemented with ferrous sulfate and cysteine, which can be transported into mitochondria (Philpott and Protchenko, Eukaryotic Cell 7: 20-27 (2008); Lee et al., Plant and Cell Physiology 55: 64-73 (2014)). Addition of ferrous sulfate was not beneficial for lipoic acid production (11.3 μg/L). In contrast, supplementation with cysteine increased lipoic acid production to 29.2 μg/L, representing almost 2.9-fold increase in titer over that from the base strain BY4741-mEfLPA. This result suggests that cysteine provides sulfur for iron-sulfur cluster biogenesis and utilization by the lipoyl synthase Lip5p to insert sulfur atoms into the carbon chain of the octanoyl group.

While we have identified a few rate-limiting steps in the lipoic acid production pathway, there is still much space for improvement to enhance lipoic acid production. To further boost the titer of lipoic acid, ion-sulfur cluster biogenesis and SAM availability, which are limiting factors of lipoic acid bio-production, can further be engineered in the future. In addition, to generate a molecule of lipoic acid, a molar equivalent of the precursor octanoyl-ACP is required (FIG. 7A). Therefore, methods to increase octanoyl-ACP supply can be explored to improve lipoic acid production. Moreover, since all the reactions take place in the mitochondria, strain engineering to increase the population of the organelle (Visser W. et al., Antonie van leeuwenhoek 67: 243-253 (1995)) can be another potential approach to increase lipoic acid titer (Zhou et al., Journal of the American Chemical Society 138: 15368-15377 (2016)). More studies are needed to resolve the bottlenecks in the lipoic acid biosynthesis pathway to markedly increase the production level. Further improvement in lipoic acid biosynthesis in yeast may be accelerated in future with rapid advances in synthetic biology and synthetic genomics for S. cerevisiae, which will offer novel tools for engineering yeast to acquire beneficial characteristics and serve as superior microbial cells factories (Chen et al., Biotechnology Advances 36: 1870-1881 (2018); Jee and Chang, Nature 557: 647-648 (2018); Xia et al., Biotechnol Adv 37: 107393 (2019)).

### SUMMARY

In this study, we aimed to develop a bio-based method for environmentally friendly lipoic acid production by metabolic engineering of S. cerevisiae. To achieve this goal, we sought to (i) understand the lipoylation process in S. cerevisiae, (ii) characterize the function of EfLPA towards lipoylated proteins from yeast, (iii) employ EfLPA to enable S. cerevisiae to produce free lipoic acid in vivo and (iv) improve lipoic acid production using metabolic engineering strategies. We first confirmed the presence of protein-bound lipoate through LC-MS/MS. Using homology modelling techniques, the protein structure of Gcv3p, Kgd2p and Lat1p were predicted and the residues for modification were found to be solvent-exposed, and hence accessible to enzymes acting on these residues. Through in vitro activity analysis, EfLPA was validated to release lipoic acid from yeast lipoyl-Gcv3p, hence demonstrating the first reported functional expression of EfLPA in yeast for releasing lipoic acid from lipoate-bound yeast protein. Subsequently, overexpression of EfLPA in the mitochondria led to lipoic acid production in vivo, thus accomplishing unprecedented free lipoic acid biosynthesis in the yeast S. cerevisiae. To enhance lipoic acid production, metabolic engineering approaches, including overexpression of pathway enzymes and regeneration of cofactors, were employed and the titer of lipoic acid production in S. cerevisiae was boosted by nearly 2.9-fold to 29.2 μg/L. Collectively, the protein analysis, enzyme characterization, structure modeling and combinatorial metabolic engineering approaches in this study provided a better understanding of the lipoic acid production pathway and revealed strategies to improve it. We envisage that the knowledge gained from this study will provide insights on lipoic acid biosynthesis in S. cerevisiae and spearhead future efforts in lipoic acid production in yeast.

### REFERENCES

Balkenhohl, F., and Paust, J. (1999). A short and productive synthesis of racemic alpha—lipoic acid. Zeitschrift Fur Naturforschung Section B-a Journal of Chemical Sciences 54, 649-654. doi: 10.1515/znb-1999-0513.
Blaschke, G., Scheidemantel, U., Bethge, H., Moller, R., Beisswenger, T., and Huthmacher, K. (1994). Preparation and use of salts of the pure enantiomers of alpha-lipoic acid. U.S. Pat. No. 5,281,722A.
Booker, S. J. (2004). Unraveling the Pathway of Lipoic Acid Biosynthesis. Chemistry & Biology 11, 10-12. doi: 10.1016/j.chembiol.2004.01.002.
Boubekeur, S., Bunoust, O., Camougrand, N., Castroviejo, M., Rigoulet, M., and Gu6rin, B. (1999). A Mitochondrial Pyruvate Dehydrogenase Bypass in the Yeast Saccharomyces cerevisiae. Journal of Biological Chemistry 274, 21044-21048. doi: 10.1074/jbc.274.30.21044.
Philpott, C. C. and Protchenko O. (2008). Response to Iron Deprivation in Saccharomyces cerevisiae. Eukaryotic Cell 7, 20-27. doi: 10.1 128/EC.00354-07.
Chen, B., Lee, D.-Y., and Chang, M. W. (2015). Combinatorial metabolic engineering of Saccharomyces cerevisiae for terminal alkene production. Metabolic Engineering 31, 53-61. doi: 10.1016/j.ymben.2015.06.009.
Chen, B., Lee, H. L., Heng, Y. C., Chua, N., Teo, W. S., Choi, W. J., Leong, S. S. J., Foo, J. L., and Chang, M. W. (2018). Synthetic biology toolkits and applications in Saccharomyces cerevisiae. Biotechnology Advances 36, 1870-1881. doi: 10.1016/j.biotechadv.2018.07.005.
Chen, B., Ling, H., and Chang, M. W. (2013). Transporter engineering for improved tolerance against alkane biofuels in Saccharomyces cerevisiae. Biotechnology for Biofuels 6, 21.
Chng, H. T., New, L. S., Neo, A. H., Goh, C. W., Browne, E. R., and Chan, E. C. Y. (2010). A sensitive LC/MS/MS bioanalysis assay of orally administered lipoic acid in rat blood and brain tissue. Journal of Pharmaceutical and Biomedical Analysis 51, 754-757. doi: 10.1016/j.jpba.2009.09.028.
Christensen Q. H., Martin N., Mansilla M. C., De Mendoza D., Cronan J. E. (2011). A novel amidotransferase required for lipoic acid cofactor assembly in Bacillus subtilis. Mol Microbiol 80, 350-363. doi: 10.1111/j.1365-2958.2011.07598.x.
Lee, C. P., Wirtz, M., Hell, R. (2014). Evidence for Several Cysteine Transport Mechanisms in the Mitochondrial Membranes of Arabidopsis thaliana. Plant and Cell Physiology 55, 64-73. doi: 10.1093/pcp/pct155.
Cicchillo, R. M., and Booker, S. J. (2005). Mechanistic Investigations of Lipoic Acid Biosynthesis in Escherichia coli: Both Sulfur Atoms in Lipoic Acid are Contributed by the Same Lipoyl Synthase Polypeptide. Journal of the American Chemical Society 127, 2860-2861. doi: 10.1021/ja042428u.
Cicchillo, R. M., Iwig, D. F., Jones, A. D., Nesbitt, N. M., Baleanu-Gogonea, C., Souder, M. G., Tu, L., and Booker, S. J. (2004). Lipoyl Synthase Requires Two Equivalents of S-Adenosyl-1-methionine To Synthesize One Equivalent of Lipoic Acid. Biochemistry 43, 6378-6386. doi: 10.1021/bi049528x.
Croce, C. D., Bronzetti, G., Cini, M., Caltavuturo, L., and Poi, G. (2003). Protective effect of lipoic acid against hydrogen peroxide in yeast cells. Toxicology in Vitro 17, 753-759. doi: 10.1016/j.tiv.2003.06.001.
Cronan, J. E. (2016). Assembly of Lipoic Acid on Its Cognate Enzymes: an Extraordinary and Essential Biosynthetic Pathway. Microbiology and Molecular Biology Reviews 80, 429-450. doi: 10.1 128/mmbr.00073-15.
Cronan, J. E., Zhao, X., and Jiang, Y. (2005). “Function, Attachment and Synthesis of Lipoic Acid in Escherichia coli,” in Advances in Microbial Physiology, ed. R. K. Poole. Academic Press), 103-146.
Dato, L., Berterame, N. M., Ricci, M. A., Paganoni, P., Palmieri, L., Porro, D., and Branduardi, P. (2014). Changes in SAM2 expression affect lactic acid tolerance and lactic acid production in Saccharomyces cerevisiae. Microbial Cell Factories 13, 147. doi: 10.1186/s12934-014-0147-7.
Dicarlo, J. E., Norville, J. E., Mali, P., Rios, X., Aach, J., and Church, G. M. (2013). Genome engineering in Saccharomyces cerevisiae using CRISPR-Cas systems. Nucleic Acids Research 41, 4336-4343. doi: 10.1093/nar/gkt135.
Foo, J. L., Susanto, A. V., Keasling, J. D., Leong, S. S. J., and Chang, M. W. (2017). Whole-cell biocatalytic and de novo production of alkanes from free fatty acids in Saccharomyces cerevisiae. Biotechnology and Bioengineering 114, 232-237. doi: 10.1002/bit.25920.
Gey, U., Czupalla, C., Hoflack, B., Krause, U., and R6del, G. (2014). Proteomic Analysis Reveals a Novel Function of the Kinase Sat4p in Saccharomyces cerevisiae Mitochondria. PLOS ONE 9, e103956. doi: 10.1371/journal.pone.0103956.
Hermes, F. A., and Cronan, J. E. (2013). The Role of the Saccharomyces cerevisiae Lipoate Protein Ligase Homologue, Lip3, in Lipoic Acid Synthesis. Yeast (Chichester, England) 30, 415-427. doi: 10.1002/yea.2979.
Hiltunen, J. K., Autio, K. J., Schonauer, M. S., Kursu, V. a. S., Dieckmann, C. L., and Kastaniotis, A. J. (2010). Mitochondrial fatty acid synthesis and respiration. Biochimica et Biophysica Acta (BBA) —Bioenergetics 1797, 1195-1202. doi: 10.1016/j.bbabio.2010.03.006.
Ide, T., Azechi, A., Suzuki, N., Kunimatsu, Y., Nakajima, C., and Kitade, S. (2013). Effects of dietary α-lipoic acid enantiomers on hepatic fatty acid metabolism in rats. Journal of Functional Foods 5, 71-79. doi: 10.1016/j.jff.2012.08.005.
Foo, J. L., and Chang, M. W. (2018). Synthetic yeast genome reveals its versatility. Nature 557, 647-648. doi: 10.1038/d41586-018-05164-3.
Ji, J.-H., Yu, I.-S., Kim, H.-J., and Oh, D.-K. (2008). Optimization of octanoic acid and sulfur donor concentrations for lipoic acid production by Pseudomonas reptilivora. Biotechnology Letters 30, 1825-1828. doi: 10.1007/s10529-008-9769-3.
Jiang, Y., and Cronan, J. E. (2005). Expression Cloning and Demonstration of Enterococcus faecalis Lipoamidase (Pyruvate Dehydrogenase Inactivase) as a Ser-Ser-Lys Triad Amidohydrolase. Journal of Biological Chemistry 280, 2244-2256. doi: 10.1074/jbc.M408612200.
Jin, Z., Wong, A., Foo, J. L., Ng, J., Cao, Y. X., Chang, M. W., and Yuan, Y. J. (2016). Engineering Saccharomyces cerevisiae to produce odd chain-length fatty alcohols. Biotechnol Bioeng 113, 842-851. doi: 10.1002/bit.25856.
Kilic, F., Handelman, G. J., Serbinova, E., Packer, L., and Trevithick, J. R. (1995). Modelling cortical cataractogenesis 17: in vitro effect of α-lipoic acid on glucose-induced lens membrane damage, a model of diabetic cataractogenesis. Biochem Mol Biol Int 37, 361-370.
Lee, W. J., Song, K.-H., Koh, E. H., Won, J. C., Kim, H. S., Park, H.-S., Kim, M.-S., Kim, S.-W., Lee, K.-U., and Park, J.-Y. (2005). α-Lipoic acid increases insulin sensitivity by activating AMPK in skeletal muscle. Biochemical and Biophysical Research Communications 332, 885-891. doi: 10.1016/j.bbrc.2005.05.035.
Li, B. J., Hao, X. Y., Ren, G. H., and Gong, Y. (2015). Effect of lipoic acid combined with paclitaxel on breast cancer cells. Genetics and molecular research 14, 17934-17940. doi: 10.4238/2015.December.22.18.
Lill, R., Dutkiewicz, R., Elsssser, H.-P., Hausmann, A., Netz, D. J. A., Pierik, A. J., Stehling, O., Urzica, E., and Muhlenhoff, U. (2006). Mechanisms of iron-sulfur protein maturation in mitochondria, cytosol and nucleus of eukaryotes. Biochimica et Biophysica Acta (BBA) —Molecular Cell Research 1763, 652-667. doi: 10.1016/j.bbamcr.2006.05.011.
Maarse, A. C., Van Loon, A. P., Riezman, H., Gregor, I., Schatz, G., and Grivell, L. A. (1984). Subunit IV of yeast cytochrome c oxidase: cloning and nucleotide sequencing of the gene and partial amino acid sequencing of the mature protein. The EMBO Journal 3, 2831-2837.
Marcelino, A. M. C., and Gierasch, L. M. (2008). Roles of p-turns in protein folding: From peptide models to protein engineering. Biopolymers 89, 380-391. doi: doi:10.1002/bip.20960.
Marobbio, C. M. T., Agrimi, G., Lasorsa, F. M., and Palmieri, F. (2003). Identification and functional reconstitution of yeast mitochondrial carrier for S-adenosylmethionine. The EMBO Journal 22, 5975-5982. doi: 10.1093/emboj/cdg574.
Martin-Perez, M., and Vill6n, J. (2017). Determinants and Regulation of Protein Turnover in Yeast. Cell systems 5, 283-294.e285. doi: 10.1016/j.cels.2017.08.008.
Marvin, M. E., Williams, P. H., and Cashmore, A. M. (2001). The isolation and characterisation of a Saccharomyces cerevisiae gene (LIP2) involved in the attachment of lipoic acid groups to mitochondrial enzymes. FEMS Microbiology Letters 199, 131-136. doi: 10.1111/j.1574-6968.2001.tb10663.x.
Moon, H.-J., Jeya, M., Yu, I.-S., Ji, J.-H., Oh, D.-K., and Lee, J.-K. (2009). Chaperone-aided expression of LipA and LpIA followed by the increase in α-lipoic acid production. Applied microbiology and biotechnology 83, 329-337. doi: 10.1007/s00253-009-1899-6.
Nagarajan, L., and Storms, R. K. (1997). Molecular Characterization of GCV3, the Saccharomyces cerevisiae Gene Coding for the Glycine Cleavage System Hydrogen Carrier Protein. Journal of Biological Chemistry 272, 4444-4450. doi: 10.1074/jbc.272.7.4444.
Piper, M. D. W., Hong, S.-P., EiBing, T., Sealey, P., and Dawes, I. W. (2002). Regulation of the yeast glycine cleavage genes is responsive to the availability of multiple nutrients. FEMS Yeast Research 2, 59-71. doi: doi:10.1111/j.1567-1364.2002.tb00069.x.
Purude, A. N., Pawar, K. P., Patil, N. B., Kalkote, U. R., and Chavan, S. P. (2015). Total synthesis of (R)-lipoic acid and (S)-lipoic acid via an Mn (Ill)-salen-catalyzed oxidative kinetic resolution. Tetrahedron-Asymmetry26, 281-287. doi: 10.1016/j.tetasy.2015.02.001.
Repetto, B., and Tzagoloff, A. (1991). In vivo assembly of yeast mitochondrial alpha-ketoglutarate dehydrogenase complex. Molecular and Cellular Biology 11, 3931-3939.
Sadowski, I., Su, T.-C., and Parent, J. (2007). Disintegrator vectors for single-copy yeast chromosomal integration. Yeast 24, 447-455. doi: 10.1002/yea.1469.
Schonauer, M. S., Kastaniotis, A. J., Kursu, V. a. S., Hiltunen, J. K., and Dieckmann, C. L. (2009). Lipoic Acid Synthesis and Attachment in Yeast Mitochondria. Journal of Biological Chemistry 284, 23234-23242. doi: 10.1074/jbc.M109.015594.
Schrödinger, Llc (2015). “The PyMOL Molecular Graphics System, Version 1.8”.).
Sinclair, D. A., and Dawes, I. W. (1995). Genetics of the synthesis of serine from glycine and the utilization of glycine as sole nitrogen source by Saccharomyces cerevisiae. Genetics 140, 1213-1222.
Spalding, M. D., and Prigge, S. T. (2009). The Amidase Domain of Lipoamidase Specifically Inactivates Lipoylated Proteins In Vivo. PLOS ONE 4, e7392. doi: 10.1371/journal.pone.0007392.
Storm J, M. S. (2012). Lipoic acid metabolism of Plasmodium--a suitable drug target. Curr Pharm Des 18, 3480-3489. doi: doi:10.2174/138161212801327266.
Stuart, B., Changkyu, O., Ursula, H., and Eckhart, S. (1997). Mitochondrial acyl carrier protein is involved in lipoic acid synthesis in Saccharomyces cerevisiae. FEBS Letters 408, 217-220. doi: doi:10.1016/S0014-5793(97)00428-6.
Sulo, P., and Martin, N. C. (1993). Isolation and characterization of LIP5. A lipoate biosynthetic locus of Saccharomyces cerevisiae. Journal of Biological Chemistry 268, 17634-17639.
Sun, Y., Zhang, W., Ma, J., Pang, H., and Wang, H. (2017). Overproduction of α-Lipoic Acid by Gene Manipulated Escherichia coli. PloS one 12, e0169369-e0169369. doi: 10.1371/journal.pone.0169369.
Villani, F., Nardi, A., Falabella, A., and Falabella, G. (2005). Synthesis of r(+)alpha-lipoic acid. U.S. Pat. No. 6,864,374B2.
Villani, F., Nardi, A., Salvi, A., and Falabella, G. (2003). Process for the production of r(+)α-lipoic acid U.S. Pat. No. 6,670,484B2.
Waterhouse, A., Bertoni, M., Bienert, S., Studer, G., Tauriello, G., Gumienny, R., Heer, F. T., De beer, T. A p., Rempfer, C., Bordoli, L., Lepore, R., and Schwede, T. (2018). SWISS-MODEL: homology modelling of protein structures and complexes. Nucleic Acids Research 46, W296-W303. doi: 10.1093/nar/gky427.
Visser W., Van Spronsen E. A., Nanninga N., Pronk J. T., Kuenen J. G., Van Dijken J. P. (1995). Effects of growth conditions on mitochondrial morphology in Saccharomyces cerevisiae. Antonie van leeuwenhoek 67, 243-253.
Xia, P.-F., Zhang, G.-C., Walker, B., Seo, S.-O., Kwak, S., Liu, J.-J., Kim, H., Ort, D. R., Wang, S.-G., and Jin, Y.-S. (2017). Recycling Carbon Dioxide during Xylose Fermentation by Engineered Saccharomyces cerevisiae. ACS Synthetic Biology 6, 276-283. doi: 10.1021/acssynbio.6b00167.
Xia, P. F., Ling, H., Foo, J. L., and Chang, M. W. (2019). Synthetic genetic circuits for programmable biological functionalities. Biotechnol Adv 37, 107393. doi: 10.1016/j.biotechadv.2019.04.015.
Zhang, J., Xin, L., Shan, B., Chen, W., Xie, M., Yuen, D., Zhang, W., Zhang, Z., Lajoie, G. A., and Ma, B. (2012). PEAKS DB: De Novo Sequencing Assisted Database Search for Sensitive and Accurate Peptide Identification. Molecular & Cellular Proteomics: MCP 11, M111.010587. doi: 10.1074/mcp.M111.010587.
Zhou, Y. J., Buijs, N. A., Zhu, Z., G6mez, D. O., Boonsombuti, A., Siewers, V., and Nielsen, J. (2016). Harnessing Yeast Peroxisomes for Biosynthesis of Fatty-Acid-Derived Biofuels and Chemicals with Relieved Side-Pathway Competition. Journal of the American Chemical Society 138, 15368-15377. doi: 10.1021/jacs.6b07394.

1. An isolated genetically engineered bacteria or yeast cell,
wherein the cell has been transformed by at least one polynucleotide molecule;
the at least one polynucleotide molecule comprising lipoic acid pathway genes which encode an octanoyltransferase, a lipoyl synthase, a protein substrate that is lipoylated, a lipoamidase and/or an S-adenosylmethionine synthase, operably linked to at least one promoter,
wherein at least one lipoic acid pathway gene is heterologous and said genetically engineered bacteria or yeast cell is capable of increased production of free lipoic acid compared to a non-transformed cell.

2. The isolated genetically engineered bacteria or yeast cell of claim 1, wherein the protein substrate that is lipoylated is selected from a group comprising Gcv3p (H protein of the glycine cleavage system), Lat1p and Kgd2p.

3. The isolated genetically engineered bacteria or yeast cell of claim 1, wherein the S-adenosylmethionine synthase is from a cell selected from a group comprising Kluyveromyces, Candida, Pichia, Yarrowia, Debaryomyces, Saccharomyces spp., and Schizosaccharomycespombe.

4. The isolated genetically engineered bacteria or yeast cell of claim 1, wherein the lipoic acid pathway genes comprise at least one gene selected from the group consisting of: LIP2 (octanoyltransferase), LIP5 (lipoyl synthase), GCV3 (H protein of the glycine cleavage system), LPA (lipoamidase), SAM1 and/or SAM2.

5. The isolated genetically engineered bacteria or yeast cell of claim 1, wherein the lipoic acid pathway genes are expressed in mitochondria.

6. The isolated genetically engineered bacteria or yeast of claim 2, wherein the lipoic acid pathway genes are expressed in the mitochondria by virtue of a mitochondrial targeting peptide (MTP).

7. The isolated genetically engineered bacteria or yeast of claim 6, wherein the mitochondrial targeting peptide (MTP) for LPA, Sam1 and/or Sam2 is from yeast cytochrome c oxidase subunit IV (COX4).

8. The isolated genetically engineered yeast of claim 1, wherein the yeast is selected from a group comprising Kluyveromyces, Candida, Pichia, Yarrowia, Debaryomyces, Saccharomyces spp., and Schizosaccharomyces pombe.

9. The isolated genetically engineered bacteria or yeast of claim 1, wherein said at least one promoter is a constitutive promoter.

10. The isolated genetically engineered bacteria or yeast of claim 1, wherein said lipoic acid pathway genes are expressed from one or more plasmids.

11. The isolated genetically engineered bacteria or yeast of claim 1, wherein at least one of said lipoic acid pathway genes is integrated into the bacteria or yeast genome.

12. The isolated genetically engineered bacteria or yeast of claim 1, wherein the lipoamidase is from Enterococcus faecalis (EfLPA).

13. The isolated genetically engineered bacteria or yeast of claim 4, wherein the LIP2, LIP5, GCV3, LPA, SAM1 and/or SAM2 genes respectively encode an amino acid sequence comprising SEQ ID NO: 1, SEQ ID NO: 3, SEQ ID NO: 5, SEQ ID NO: 7, SEQ ID NO: 9 and/or SEQ ID NO: 11.

14. The isolated genetically engineered bacteria or yeast of claim 4, wherein the LIP2, LIP5, GCV3, LPA, SAM1 and/or SAM2 genes respectively comprises a polynucleotide sequence having at least 70% sequence identity, at least 80% sequence identity, at least 90% sequence identity or 100% sequence identity to SEQ ID NO: 2, SEQ ID NO: 4, SEQ ID NO: 6, SEQ ID NO: 8, SEQ ID NO: 10 and/or SEQ ID NO: 12.

15. A recombinant expression vector comprising one or more heterologous lipoic acid pathway genes defined in claim 1, operably linked to a promoter, wherein an expressed protein from said pathway genes is located to the mitochondria.

16. The recombinant vector of claim 15, wherein said promoter is a constitutive promoter.

17. A method of producing free lipoic acid in a genetically engineered cell, comprising the steps:
a) culturing a plurality of genetically engineered cells of claim 1 in medium under conditions for lipoic acid biosynthesis, and
b) supplementing the medium with cysteine,
wherein said genetically engineered cell is capable of increased production of free lipoic acid compared to a non-transformed cell.

18. The method of claim 17, wherein the medium is supplemented with cysteine at a concentration of at least 0.05 mg/ml, at least 0.1 mg/ml, at least 0.2 mg/ml, at least 0.5 mg/ml or in the range from 0.05 mg/ml to 0.7 mg/ml, preferably in the range 0.1 mg/ml to 0.4 mg/ml.

19. The method of claim 17, further comprising isolating said free lipoic acid.

20. The method of claim 17, wherein the engineered cell is a yeast cell.

21. The method of claim 20, wherein the engineered cell is Saccharomyces cerevisiae.


SEQUENCE LISTING (45 sequences)

SEQ ID NO: 1 - protein, 334 aa, Artificial Sequence
  Lip2
  MISC_FEATURE (328)..(344): 6x histidine tag
MSRCIRQSVCTNFNVCRRQCFSTYASALKEMTHPIKPSAQTLRHLQFTQRIPFQKGLEIQETLVRANLDIKDIQSKIERKLIQLDEEYKGTATINDNEKRILDKVMAMKPNPIILTFEFEPTYTGGKRIKKTMTPDQIAAYESFIPETQKDNPRPKFVQVERGGQVTFHGPGQIVIYIILDLKTFQSFPAKCLVSCIEQATIRTLKNTKMCDDTDKPLNLDAMTTKDTGVWVENGKKKVASVGIHVRRSITSHGVAINVNTDLSYMNSFEMCGLKNTLTTSIMEQRPDAVVNVQSVAISFVKEMTKLLGIKTLERMQIDDVNILKKNPHHHHHH

SEQ ID NO: 2 - DNA, 1005 bp, Artificial Sequence
  LIP2
  misc_feature (985)..(1005): 6x histidine tag
atgtcgaggtgtattcgccagtccgtatgcacaaacttcaatgtctgtaggcgacaatgctttagcacttatgcctccgctctcaaggaaatgacacatcctataaaaccctcagcgcaaacgctcagacatctacaattcactcaaagaataccttttcaaaaaggattggaaatacaggagacattagttagggcaaatttggacatcaaagacattcaatcgaagattgagagaaaattaatacaattggatgaagaatataagggcactgcaacaataaatgataacgaaaaaagaatattagacaaggttatggcaatgaaacctaatccgataatattaacgtttgagtttgagccaacttatacaggtgggaagaggattaagaaaactatgacgccagatcagatagcggcttatgaaagtttcattccagaaacgcaaaaggacaacccacgaccaaagtttgttcaagtggaaaggggtggacaagtaacatttcatggtccaggccaaattgttatttacattattttagatttgaaaacatttcaaagtttccctgctaaatgcttggtttcatgcattgagcaggccactatcagaacgttgaagaataccaagatgtgcgacgacactgacaagcctttgaacttagatgctatgactacaaaggacactggagtttgggttgaaaatggcaagaagaaagttgctagtgttggaatacacgtcagacgttccataacatcgcacggggtagcaattaatgttaatacagatctatcgtatatgaatagttttgaaatgtgcggtttgaaaaatacactgactacttcaattatggaacaacgacccgatgctgttgtaaatgtgcaaagtgtagcaattagttttgtcaaagaaatgacaaagctgctaggaataaaaactttggaaagaatgcaaattgacgacgtcaatattttgaaaaagaatccgcatcatcaccatcaccattaa

SEQ ID NO: 3 - protein, 420 aa, Artificial Sequence
  Lip5
  MISC_FEATURE (415)..(420): 6x histidine tag
MYRRSVGVLFVGRNTRWISSTIRCGTSATRPIRSNALNTDSDNASVRVPVGNSTEVENATSQLTGTSGKRRKGNRKRITEFKDALNLGPSFADFVSGKASKMILDPLEKARQNTEEAKKLPRWLKVPIPKGTNYHKLKGDVKELGLSTVCEEARCPNIGECWGGKDKSKATATIMLLGDTCTRGCRFCSVKTNRTPSKPDPMEPENTAEAIKRWGLGYVVLTTVDRDDLVDGGANHLAETVRKIKQKAPNTLVETLSGDFRGDLKMVDIMAQCGLDVYAHNLETVESLTPHVRDRRATYRQSLSVLERAKATVPSLITKTSIMLGLGETDEQITQTLKDLRNIQCDVVTFGQYMRPTKRHMKVVEYVKPEKFDYWKERALEMGFLYCASGPLVRSSYKAGEAFIENVLKKRNMKHHHHHH

SEQ ID NO: 4 - DNA, 1263 bp, Artificial Sequence
  LIP5
  misc_feature (1243)..(1263): 6x histidine tag
atgtatagacgatctgttggagtactatttgttgggagaaatacaagatggatttcgtccactattaggtgtggaacgagcgcaactcggccaataagatccaatgcattgaatactgattcagataacgctagcgttagggtaccagttggaaattcaactgaggttgaaaatgcaaccagtcagttaacagggacttcgggaaaaagacgcaaggggaacagaaaaagaattacagagttcaaagatgctcttaatttgggtccatcttttgcagattttgtttcaggtaaagcttcgaaaatgatcttggaccctctggagaaggcaaggcaaaatacagaggaagctaaaaaactacctcgttggcttaaggttcctattcctaagggtaccaattaccataaattgaaaggcgacgtaaaagaattaggcctgagtactgtttgtgaggaggcaagatgtcccaatattggtgaatgttggggaggcaaagataaatctaaggcaacggcaacaattatgctgcttggtgatacttgcactcgtggatgtaggttttgttctgtgaagaccaatagaacgcctagtaagccggacccaatggagcccgaaaatactgccgaagctatcaaaagatgggggttgggttatgttgttttaactaccgttgatagggacgatttagtcgatggtggtgctaatcacctggccgaaacggttcgcaaaatcaaacagaaggcaccaaatactcttgtagagactctttctggtgatttcagaggtgatttgaagatggtggacattatggcacaatgtgggcttgatgtttacgcacataatttggaaacagttgaatcactaacaccacatgtcagagacagaagagctacttatagacagtctttgagtgttttagaaagggcaaaagctacggttccgtcactgattactaaaacatcaataatgctgggtctaggagaaactgatgagcaaattactcaaactttgaaggatttgcgcaatattcaatgtgatgttgttacatttggtcaatatatgaggccaaccaagagacatatgaaagtcgtagaatatgtgaaacccgaaaagtttgactactggaaagagagagctttagagatgggattcttgtattgtgcatctggacccctagtaagatcgtcatataaggctggtgaagcatttattgagaacgttttgaagaaaagaaacatgaaacatcatcaccatcaccattaa

SEQ ID NO: 5 - protein, 176 aa, Artificial Sequence
  Gcv3
  MISC_FEATURE (171)..(176): 6x histidine tag
MLRTTRLWTTRMPTVSKLFLRNSSGNALNKNKLPFLYSSQGPQAVRYTSQHEWIAVHQDKTAFVGITKYATDALGDATYVELPEVGTEIAQGESLGSIESVKSASEIYQPADGTVEEINTNLEENPGVVNEDPMGDGWLVKMKLGEGVNVEQVEGLMSLEQYEKTLVHDDHHHHHH

SEQ ID NO: 6 - DNA, 531 bp, Artificial Sequence
  GCV3
  misc_feature (511)..(531): 6x histidine tag
atgttacgcactactagactatggaccacccgcatgcccactgtgagcaaattgtttttgagaaacagctccggcaatgccctaaacaagaataaactaccatttttgtactcatcccaaggacctcaagccgtgaggtacacttcccaacatgagtggatagctgtgcatcaggacaagactgcctttgtcggaattacaaaatacgccactgatgccttaggggacgctacctatgttgagttgccagaagtgggcactgagattgcccaaggtgagtcgctagggtccattgagtccgtcaagtcagcctccgagatctaccagcctgccgatggtaccgtagaggaaattaacactaatcttgaggaaaatccaggtgtggtgaacgaagatcctatgggtgacggctggctagtcaaaatgaagcttggtgagggcgttaatgtggaacaggtcgagggtctaatgtccttagaacagtacgaaaagacactggttcatgatgaccatcatcaccatcaccattga

SEQ ID NO: 7 - protein, 760 aa, Artificial Sequence
  Lpa
  MISC_FEATURE (755)..(760): 6x histidine tag
MLSLRQSIRFFKPATRTLCSSRYLLQQKPMLAQESILETTVQTETESVTTETSQTVANLESETTSQTVMQEKESSSAIAESSSRNVVAVTTETTNEIQNSGTDGKAVSAESVFSEADYKQATALELATLVREKKVTSEELVKIALAITKRENPTLNAVITLREEAALTEAKALQDTGQPFLGVPLLLKGLGQSLKGESNTNGFGFLRDQVAGGTSTFVKALQNAGFIIIGQTNYPELGWKNISDSKLYGVSVNPWNPNHYSGGSSGGAGASVAAAFVPIASGSDAGGSIRIPASWTGTVGLKPSRGVIIGNSNSAKGQTVHFGLSRTVADTNALFETLLTKKDLPAGHLSQAQPIAYTTESPAGTPVSAEAKEAVAEAVAFLKDQGYTLVEVKHPVDGERLMKNYYTVAAGSAGIADFMARQKLKRPLERNDVELLTWALFQTGKNITSEETTAAWTDIALQAQAMDEFYQQYPILLTPTTAATAPSIDNPLLKPEHAAQMEKIDQLSPAEQKQLIYDQWLTAFTYTPFTQQANLFGHPALSVPTYVSKEGLPLGIQFNSALNEDRTLLQLGALFENNHKINQPHVEEPDKDKEPDASGEPEKDKDPNASGEPDKDKEPDASGEPDKDKEPDASGEPDKDKEPDASGKPDKDKETKTSEGPIEGKDQNQNPDKAGKTTSGSSLDNSLNSSANQGTKSTESTHAFSNKSMIGKQEQLPKKVLPKAGAEVPSTFWIVLGGAFLVTSGTIYIRKTRKHHHHHH

SEQ ID NO: 8 - DNA, 2283 bp, Artificial Sequence
  LPA
  misc_feature (1)..(87): MTP
  misc_feature (2263)..(2283): 6x histidine tag
atgctttcactacgtcaatctataagatttttcaagccagccacaagaactttgtgtagctctagatatctgcttcagcaaaaacccatgctagcccaagaaagcattttggaaacaacagtacagactgaaacagagagtgtgactacggagacaagccagacagtcgccaacttggagtctgagactaccagccagactgtaatgcaagaaaaagagagttcttcagctatcgcagaaagctcatcaagaaatgtggttgcggtaacaactgaaaccacgaatgagattcaaaatagtggtactgacgggaaggctgtgtcagctgaatctgtattttctgaagcagattacaaacaagctactgcattagaacttgctactcttgttagagagaaaaaggtcacttccgaggaattagttaagatcgctctagccatcactaaacgtgagaatccaacccttaatgccgtcattactttaagagaagaggcagctttaacggaggccaaggccttgcaagacactggacaaccgtttttaggagttcctttgttgttgaagggtttgggtcagtcattaaagggagaatctaacacgaatggcttcggttttctaagggaccaagtggctggtggaacttcaactttcgttaaagcattgcagaacgcgggattcatcataattggccaaactaattatccggaactaggctggaaaaatatatcagactcaaagttatatggggtgtccgtcaatccatggaatcccaaccattattccggaggtagtagtgggggagcgggtgctagtgtggccgcagcattcgtccccattgctagtggttccgatgctggtggctcaattagaatacctgcgagttggactggcactgtgggactgaaaccatctcgtggggttattattggcaattctaactcagccaagggtcaaacggtccatttcggtttaagcagaacagttgccgacactaatgcgcttttcgaaactcttcttaccaagaaggatttgccagctggccatttatcccaagcacaacccatagcgtacactaccgaaagtccagcaggtacacccgtaagcgctgaggccaaagaggctgtggccgaagcagtagcttttttaaaggaccaaggctacactttagttgaagttaagcatcccgttgacggtgaaagacttatgaaaaactattacacggtcgccgctggtagtgccggcatcgccgactttatggccaggcagaagctaaagcgtcctttagagagaaacgacgtcgaactgctaacatgggcgttgttccaaacagggaaaaatataacaagtgaagaaactacagccgcatggaccgatatcgctctacaagcccaagccatggatgaattttatcaacaatatccaattttgttaacaccgacgaccgcagctaccgcacccagtattgataatccgttattgaagccagaacatgccgcccaaatggaaaagatcgatcagttatctccggctgaacagaagcaattaatttatgatcaatggttaactgctttcacatataccccgttcacacaacaagcgaacctattcggtcacccggctttatctgtacccacttatgtctccaaagagggtttgccactgggaattcaatttaactccgccttgaatgaagatagaactttattgcagctaggtgcattgttcgaaaacaatcacaaaatcaaccagcctcatgttgaagagcctgataaagacaaggaacctgatgccagcggagagcctgaaaaagataaggacccaaatgcgtccggtgagccagacaaggacaaagagcccgatgcttcaggtgaacccgataaagataaagaaccagatgccagtggggaaccagataaagataaggaaccagacgcaagtggtaagccagataaagataaagagaccaagacttccgaagggccgatcgagggtaaagatcaaaaccaaaatcccgataaggctggtaagacaacctcagggtctagcctagataatagtttaaatagcagtgcaaaccagggcacaaaatccactgaatcaactcatgcgttttctaataaatccatgatcggtaaacaagaacaattgccgaaaaaagtattgccaaaggcgggagcagaagtgccatctactttctggattgttcttggtggtgctttcttagtgacgagtggtactatctacattagaaagacccgtaagcatcatcaccatcaccattaa

SEQ ID NO: 9 - protein, 423 aa, Artificial Sequence
  Sam1
  MISC_FEATURE (418)..(423): 6x histidine tag
MLSLRQSIRFFKPATRTLCSSRYLLQQKPHHHHHHMAGTFLFTSESVGEGHPDKICDQVSDAILDACLAEDPHSKVACETAAKTGMIMVFGEITTKAQLDYQKIVRDTIKKIGYDDSAKGFDYKTCNVLVAIEQQSPDIAQGVHEEKDLEDIGAGDQGIMFGYATDETPEGLPLTILLAHKLNMAMADARRDGSLAWLRPDTKTQVTVEYKDDHGRWVPQRIDTVVVSAQHADEITTEDLRAQLKSEIIEKVIPRDMLDENTKYFIQPSGRFVIGGPQGDAGLTGRKIIVDAYGGASSVGGGAFSGKDYSKVDRSAAYAARWVAKSLVAAGLCKRVQVQFSYAIGIAEPLSLHVDTYGTATKSDEEIIDIISKNFDLRPGVLVKELDLARPIYLPTASYGHFTNQEYPWEKPKTLKFHHHHHH

SEQ ID NO: 10 - DNA, 1272 bp, Artificial Sequence
  SAM1
  misc_feature (1)..(87): MTP
  misc_feature (1252)..(1272): 6x histidine tag
atgctttcactacgtcaatctataagatttttcaagccagccacaagaactttgtgtagctctagatatctgcttcagcaaaaaccccatcatcaccatcaccatatggccggtacatttttattcacttctgaatccgttggtgaaggtcacccagataagatctgtgaccaagtttccgacgccatcttggacgcttgtttagccgaggaccctcactccaaagttgcgtgtgaaaccgcggcaaagactggtatgattatggtctttggtgaaattactaccaaggcacagttggattaccaaaaaatcgtcagagacaccatcaagaagattggttacgatgattccgccaagggtttcgactataagacctgtaacgtccttgtcgccattgagcaacaatctccagatatcgcccaaggtgtccacgaggagaaggatttggaagacatcggtgccggtgaccaaggtatcatgtttggttacgccacagatgaaactccagagggtttgcctttgactattcttttggctcataaactaaacatggccatggctgacgcgagaagagatggctctttagcgtggttgagaccagacaccaagactcaagtcaccgtcgaatacaaggatgaccacggtagatgggttccacaaagaatcgacaccgtcgtcgtctccgctcaacatgctgacgaaatcacgaccgaggacttaagagcgcaactaaagtccgagatcattgaaaaagtcatcccaagagacatgttggacgaaaacaccaaatactttatccaaccttccggtagattcgtcatcggtggtcctcaaggtgacgctggtttgaccggtagaaagatcatcgtcgacgcttacggtggtgcctcatccgtcggtggtggtgccttctccggtaaggactactctaaggttgatcgttctgccgcttatgccgctagatgggttgccaagtccctagttgccgctggtttatgtaagagagttcaagttcaattttcttatgccatcggtattgcggaaccattgtccttgcacgttgacacctatggtactgcgaccaagtctgacgaagaaattatcgacattatcagcaagaactttgacttgagacctggtgtattggtcaaggagttggacttagctagaccaatctacttgccaaccgcttcttatggccatttcacaaaccaagaatacccatgggaaaagcctaagactttgaagttccatcatcaccatcaccattaa

SEQ ID NO: 11 - protein, 425 aa, Artificial Sequence
  Sam2
  MISC_FEATURE (419)..(425): 6x histidine tag
MLSLRQSIRFFKPATRTLCSSRYLLQQKPHHHHHHMSKSKTFLFTSESVGEGHPDKICDQVSDAILDACLEQDPFSKVACETAAKTGMIMVFGEITTKARLDYQQIVRDTIKKIGYDDSAKGFDYKTCNVLVAIEQQSPDIAQGLHYEKSLEDLGAGDQGIMFGYATDETPEGLPLTILLAHKLNMAMADARRDGSLPWLRPDTKTQVTVEYEDDNGRWVPKRIDTVVISAQHADEISTADLRTQLQKDIVEKVIPKDMLDENTKYFIQPSGRFVIGGPQGDAGLTGRKIIVDAYGGASSVGGGAFSGKDYSKVDRSAAYAARWVAKSLVAAGLCKRVQVQFSYAIGIAEPLSLHVDTYGTATKSDDEIIEIIKKNFDLRPGVLVKELDLARPIYLPTASYGHFTNQEYSWEKPKKLEFHHHHHH

SEQ ID NO: 12 - DNA, 1278 bp, Artificial Sequence
  SAM2
  misc_feature (1)..(87): MTP
  misc_feature (1258)..(1278): 6x histidine tag
atgctttcactacgtcaatctataagatttttcaagccagccacaagaactttgtgtagctctagatatctgcttcagcaaaaaccccatcatcaccatcaccatatgtccaagagcaaaactttcttatttacctctgaatccgtcggtgaaggtcacccagacaagatttgtgaccaagtttctgatgctattttggacgcttgtttagaacaagatccattctccaaggttgcctgtgaaacagctgccaaaactggtatgattatggttttcggtgaaattaccaccaaagctagacttgactaccaacaaatagtaagagataccatcaagaagattggttatgacgattctgccaagggtttcgactacaagacatgtaatgttttagtagctatcgaacaacaatctccagatatcgctcaaggtctgcactatgaaaagagcttagaagacttaggtgctggtgaccaaggtataatgtttggttacgctacagacgaaactccagaagggttaccattgaccattcttttggctcacaaattgaacatggctatggcagatgctagaagagatggttctctcccatggttgagaccagacacaaagactcaagtcactgtcgaatacgaagacgacaatggtagatgggttccaaagaggatagataccgttgttatttctgctcaacatgctgatgaaatttccaccgctgacttgagaactcaacttcaaaaagatattgttgaaaaggtcataccaaaggatatgttagacgaaaataccaaatatttcatccaaccatccggtagattcgtcatcggtggtcctcaaggtgacgctggtttgaccggtagaaagattattgtcgacgcttacggtggtgcctcatccgtcggtggtggtgccttctccggtaaggactattccaaggtcgatcgttccgctgcttacgctgctagatgggttgccaagtctctagttgccgctggtttgtgtaagagagtccaagtccaattttcatatgctattggtattgctgaaccattgtctttacatgtggacacctatggtacagctacaaaatcagatgacgaaatcattgaaattattaagaagaacttcgacttgagaccaggtgtgttagtaaaggaattagatttggctagaccaatttacttaccaaccgcttcttatggtcacttcactaatcaagagtactcatgggaaaaaccaaagaaattggaatttcatcatcaccatcaccattaa

SEQ ID NO: 13 - DNA, 27 bp, Artificial Sequence
  PGAL1-F
aaacgagctcagtacggattagaagcc

SEQ ID NO: 14 - DNA, 27 bp, Artificial Sequence
  PGAL1-R
tttttagggttttttctccttgacgtt

SEQ ID NO: 15 - DNA, 20 bp, Artificial Sequence
  TCYC1-F
atccgctctaaccgaaaagg

SEQ ID NO: 16 - DNA, 29 bp, Artificial Sequence
  TCYC1-R
aaacgagctccttcgagcgtcccaaaacc

SEQ ID NO: 17 - DNA, 40 bp, Artificial Sequence
  EfLPA-F
cgtcaaggagaaaaaaccctaaaaaatgctagcccaagaa

SEQ ID NO: 18 - DNA, 127 bp, Artificial Sequence
  mEfLPA-F
cgtcaaggagaaaaaaccctaaaaaatgctttcactacgtcaatctataagatttttcaagccagccacaagaactttgtgtagctctagatatctgcttcagcaaaaacccatgctagcccaagaa

SEQ ID NO: 19 - DNA, 77 bp, Artificial Sequence
  EfLPA-R
ctaactccttccttttcggttagagcggattcattaatggtgatggtgatgatgcttacgggtctttctaatgtaga

SEQ ID NO: 20 - DNA, 40 bp, Artificial Sequence
  EGFP-F
cgtcaaggagaaaaaaccctaaaaaatgtctaaaggtgaa

SEQ ID NO: 21 - DNA, 127 bp, Artificial Sequence
  mEGFP-F
cgtcaaggagaaaaaaccctaaaaaatgctttcactacgtcaatctataagatttttcaagccagccacaagaactttgtgtagctctagatatctgcttcagcaaaaacccatgtctaaaggtgaa

SEQ ID NO: 22 - DNA, 69 bp, Artificial Sequence
  EGFP-R
ctaactccttccttttcggttagagcggattcattaatggtgatggtgatgatgtttgtacaattcatc

SEQ ID NO: 23 - DNA, 38 bp, Artificial Sequence
  PTEF1-F
accgctcgagcatagcttcaaaatgtttctactccttt

SEQ ID NO: 24 - DNA, 27 bp, Artificial Sequence
  PTEF1-R
ttgtaattaaaacttagattagattgc

SEQ ID NO: 25 - DNA, 51 bp, Artificial Sequence
  GCV3-F
gcaatctaatctaagttttaattacaaatgttacgcactactagactatgg

SEQ ID NO: 26 - DNA, 72 bp, Artificial Sequence
  GCV3-R
ctaactccttccttttcggttagagcggattcattaatggtgatggtgatgatggtcatcatgaaccagtgt

SEQ ID NO: 27 - DNA, 45 bp, Artificial Sequence
  KGD2-F
gcaatctaatctaagttttaattacaaatgctttccagagcgacg

SEQ ID NO: 28 - DNA, 74 bp, Artificial Sequence
  KGD2-R
atcagattggtatgggctgcaaatttcaaatcattaatggtgatggtgatgatgccataacaacatttttctag

SEQ ID NO: 29 - DNA, 21 bp, Artificial Sequence
  TKGD2-F
tttgaaatttgcagcccatac

SEQ ID NO: 30 - DNA, 39 bp, Artificial Sequence
  TKGD2-R
attcgagctcatgtggaaatcaaaagaatattagttgat

SEQ ID NO: 31 - DNA, 48 bp, Artificial Sequence
  LAT1-F
gcaatctaatctaagttttaattacaaatgtctgcctttgtcagggtg

SEQ ID NO: 32 - DNA, 74 bp, Artificial Sequence
  LAT1-R
taataaaaatcataaatcataagaaattcgtcattaatggtgatggtgatgatgcaatagcatttccaaaggat

SEQ ID NO: 33 - DNA, 27 bp, Artificial Sequence
  TADH1-F
cgaatttcttatgatttatgattttta

SEQ ID NO: 34 - DNA, 31 bp, Artificial Sequence
  TADH1-R
acgcggatccgagcgacctcatgctatacct

SEQ ID NO: 35 - DNA, 67 bp, Artificial Sequence
  LIP2-LIP5-CS6-F
aacctcgaggagaagtttttttacccctctccacagatcctcgagcatagcttcaaaatgtttctac

SEQ ID NO: 36 - DNA, 67 bp, Artificial Sequence
  LIP2-LIP5-CS6-R
taattaggtagaccgggtagatttttccgtaaccttggtgtcgagctcacgcatttttttcttttgc

SEQ ID NO: 37 - DNA, 73 bp, Artificial Sequence
  SAM1/2-CS8-F
caaaattacctacggtaattagtgaaaggccaaaatctaatgttacaatagtatactagaagaatgagccaag

SEQ ID NO: 38 - DNA, 76 bp, Artificial Sequence
  SAM1-CS8-R
gaccgttcccttgtgttgtaccagtggtagggttcttctcggtagcttctataagataaagtttggtttgttgatc

SEQ ID NO: 39 - DNA, 76 bp, Artificial Sequence
  SAM2-CS8-R
gaccgttcccttgtgttgtaccagtggtagggttcttctcggtagcttctcctcaaagacattctatatttcaacc

SEQ ID NO: 40 - protein, 7 aa, Artificial Sequence
  Fragment from Gcv3p
  MISC_FEATURE (3)..(3): lipoic acid modification
SVKSASE

SEQ ID NO: 41 - protein, 7 aa, Artificial Sequence
  Fragment from Kgd2p
  MISC_FEATURE (3)..(3): lipoic acid modification
TDKIDIE

SEQ ID NO: 42 - protein, 9 aa, Artificial Sequence
  Fragment from Lat1p
  MISC_FEATURE (3)..(3): lipoic acid modification
TDKAQMDFE

SEQ ID NO: 43 - protein, 7 aa, Artificial Sequence
  Fragment from Gcv3p
  MISC_FEATURE (1)..(1): lipoic acid modification
SVKSASE

SEQ ID NO: 44 - protein, 7 aa, Artificial Sequence
  Fragment from Gcv3p
  MISC_FEATURE (4)..(4): lipoic acid modification
SVKSASE

SEQ ID NO: 45 - protein, 29 aa, Artificial Sequence
  MTP
MLSLRQSIRFFKPATRTLCSSRYLLQQKP